(19) United States
(12) Patent Application Publication
(10) Pub. No.: US 2024/0415900 A1
(43) Pub. Date: Dec. 19, 2024

(54) CLOSTRIDIUM BUTYRICUM COMPOSITIONS AND METHODS OF USING THE SAME

(71) Applicant: City of Hope, Duarte, CA (US)

(72) Inventors: Ryotaro Nakamura, Duarte, CA (US); Karamjeet Sandhu, Duarte, CA (US); Peter P. Lee, Duarte, CA (US)

(21) Appl. No.: 18/702,614

(22) PCT Filed: Oct. 20, 2022
(86) PCT No.: PCT/US2022/047328

§ 371 Date: Apr. 18, 2024

Related U.S. Application Data
(60) Provisional application No. 63/275,331, filed on Nov. 3, 2021.
(60) Provisional application No. 63/257,993, filed on Oct. 20, 2021.

(51) Int. Cl. A61K 35/742; A61P 37/06

(52) U.S. Cl. CPC A61K 35/742; A61P 37/06

(57) ABSTRACT

Provided herein, inter alia, are methods and compositions for the treatment of graft-versus-host disease. The compositions include Clostridium butyricum. Administration of Clostridium butyricum can enhance gut biodiversity and improve clinical outcome of a patient undergoing allogeneic hematopoietic stem cell transplantation.

### CROSS REFERENCE TO RELATED APPLICATIONS

This application claims the benefit of priority to U.S. Provisional Application Ser. No. 63/257,993 filed Oct. 20, 2021 and U.S. Provisional Application Ser. No. 63/275,331 filed Nov. 3, 2021. The disclosures of these applications are incorporated herein by reference in their entirties and for all purposes.

### BACKGROUND

Allogeneic hematopoietic stem cell transplantation (HCT) is a curative treatment option for a wide range of hematological and non-hematological disorders. Despite recent advances in supportive care, graft-versus-host disease (GVHD), infection, and relapse of the underlying hematologic disease remain the major causes of morbidity and mortality in HCT recipients.

The gut microbiota has been shown to play an important role in maintaining intestinal homeostasis by regulating the maturation of the mucosal immune system, which constitutes an immune barrier for the integrity of the intestinal tract. (Shi N., et al., Mil Med Res. 2017). AlloHCT patients are a unique population in which the gut ecosystem is already perturbed by previous interventions including chemotherapy and antibacterial therapy, and then further impaired as a result of intensive conditioning regimens and donor T cell assault (GVHD). In recent years, the role of the human GI microbiota in GVHD has been considered in observational studies. (Jeng, R. R., et al., J Exp Med, 2012. 209(5): p. 903-11). However, there have been no prospective trials to date demonstrating a clinical benefit of dietary interventions specifically designed to alter the microbiota of HCT recipients.

CBM588 is a novel Live Biotherapeutic Product (LBP) that contains the bacterium Clostridium butyricum MIYAIRI 588 (CBM588). CBM588 produces short chain organic acids (SCFAs), most notably butyric acid, which plays a key role in the maintenance of colonic homeostasis by regulating fluid and electrolyte uptake, epithelial cell growth, and inflammatory responses (Hamer, H. M, et al., Aliment Pharmacol Ther, 2008. 27(2): p. 104-19). CBM588 has a spectrum of activities that also includes direct antagonism of enteric pathogens, stimulation of the growth of beneficial bacteria, including bifidobacteria and lactobacilli, and recovery of the normal anaerobic microflora following disruption of the intestinal ecosystem, typically by antibiotic use (Takahashi, M., et al., FEMS Immunol Med Microbiol, 2004. 41(3): p. 219-226); Takashi, K., et al., Jpn J Pharmacol, 1989. 50(4): p. 495-498; Okamoto, T., et al., J Gastroenterol, 2000. 35(5): p. 341-346; Shimbo, I., et al., World J Gastroenterol, 2005. 11(47): p. 7520-7524; Da-Rong, et al., Chin J Gastroenterol 1998. 3(2); Seki, H., et al., Pediatr Int, 2003. 45(1): p. 86-90; Kuroiwa, T., et al., J Jpn Association Infect Dis 1990. 64(11): p. 1425-1432).

There is a need for safe and effective treatment of GVHD in patients who underwent allogeneic hematopoietic stem cell transplantation. The disclosure below solves these and other problems in the art.

### BRIEF SUMMARY

In an aspect is provided a method for treating graft-versus-host disease in a subject in need thereof, the method comprising administering to the subject a therapeutically effective amount of Clostridium butyricum. Further provided is a method for treating graft-versus-host disease in a subject in need thereof, the method comprising administering to the subject an effective amount of Clostridium butyricum live biotherapeutic product (LBP). Further provided is a method for treating graft-versus-host disease in a subject in need thereof, the method comprising administering to the subject an effective amount of Clostridium butyricum MIYAIRI 588 live biotherapeutic product (CBM588 LBP). In embodiments, the subject is a subject who is undergoing or who has undergone allogeneic hematopoietic stem cell transplantation. In embodiments, the subject is a cancer subject who is undergoing or who has undergone allogeneic hematopoietic stem cell transplantation. In embodiments, the subject is a hematologic cancer subject. In embodiments, the subject suffers from non-cancer hematological disorder. In embodiments, the subject has a non-cancer hematological disorder. In embodiments, the subject suffers from an autoimmune disease. In embodiments, the subject has an autoimmune disease.

In an aspect is provided a method of increasing gut biodiversity in a subject in need thereof, the method comprising administering to the subject a therapeutically effective amount of Clostridium butyricum. Further provided is a method of increasing gut biodiversity in a subject in need thereof, the method comprising administering to the subject an effective amount of Clostridium butyricum live biotherapeutic product (LBP). Further provided is a method of increasing gut biodiversity in a subject in need thereof, the method comprising administering to the subject an effective amount of Clostridium butyricum MIYAIRI 588 live biotherapeutic product (CBM588 LBP). In embodiments, the subject is a subject who is undergoing or who has undergone allogeneic hematopoietic stem cell transplantation. In embodiments, the subject is a cancer subject who is undergoing or who has undergone allogeneic hematopoietic stem cell transplantation. In embodiments, the subject is a hematologic cancer subject. In embodiments, the subject suffers from non-cancer hematological disorder. In embodiments, the subject suffers from an autoimmune disease.

In an aspect is provided a method of improving clinical outcome of a patient undergoing allogeneic hematopoietic stem cell transplantation, said method comprising administering to said patient a therapeutically effective amount of Clostridium butyricum. Further provided is a method of improving clinical outcome of a patient undergoing allogeneic hematopoietic stem cell transplantation, said method comprising administering to said patient a therapeutically effective amount of Clostridium butyricum live biotherapeutic product (LBP). Further provided is a method of improving clinical outcome of a patient undergoing allogeneic hematopoietic stem cell transplantation, said method comprising administering to said patient a therapeutically effective amount of Clostridium butyricum MIYAIRI 588 live biotherapeutic product (CBM588 LBP). In embodiments, the subject is a subject who is undergoing or who has undergone allogeneic hematopoietic stem cell transplantation. In embodiments, the subject is a cancer subject who is undergoing or who has undergone allogeneic hematopoietic stem cell transplantation. In embodiments, the subject is a hematologic cancer subject. In embodiments, the subject suffers from non-cancer hematological disorder. In embodiments, the subject suffers from an autoimmune disease.

In an aspect is provided a pharmaceutical composition comprising Clostridium butyricum. Further provided is a pharmaceutical composition comprising Clostridium butyricum as a live biotherapeutic product. Further provided is a pharmaceutical composition comprising Clostridium butyricum MIYAIRI 588 as a live biotherapeutic product.

### BRIEF DESCRIPTION OF THE DRAWINGS

FIG. 1 depicts Shanon Diversity index comparison between control and treatments arms in the Randomized Open Label Pilot Study of Clostridium butyricum MIYAIRI 588 (CBM588) in Recipients of Allgeneic Hematopoiei Cell Transplantation.

FIG. 2 depicts Bray-Curtis hierarchical clustering of top thirty taxa. Groups are indicated on the top bar: Control Arm (blue) and Treatmet Arm (red). Relative abundance is colored on a log scale as indicated by the key at the top of the figure.

### DETAILED DESCRIPTION

### Definitions

An amino acid residue in a protein “corresponds” to a given residue when it occupies the same essential structural position within the protein as the given residue.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, y-carboxyglutamate, and O-phosphoserine. In embodiments, the amino acid side chain may be a non-natural amino acid side chain. In embodiments, the amino acid side

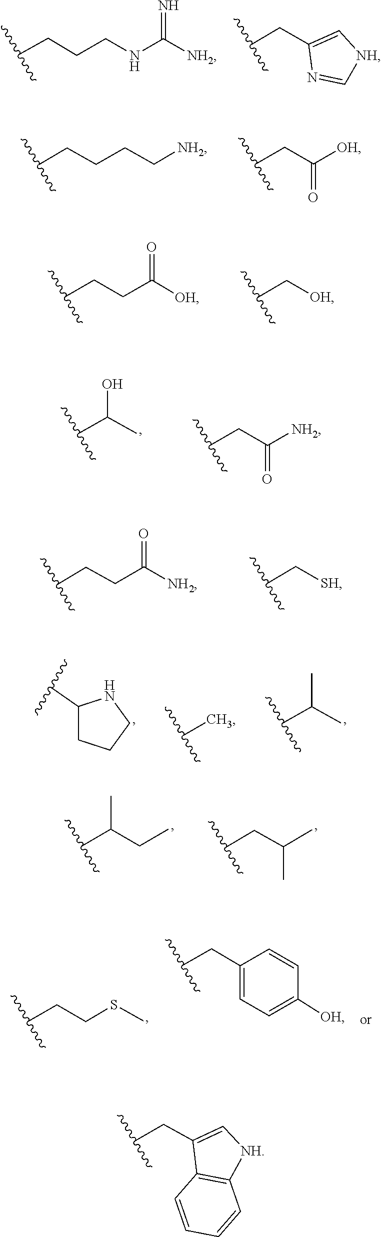

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “VL” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. The disease may be a graft-versus-host disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemias, lymphomas, myelomas, carcinomas and sarcomas.

As used herein, the term “chemotherapeutic resistant cancer” or “chemotherapeutic resistant” refers to lack of intended response of a cancer to a chemotherapy. Chemotherapeutic resistance may refer to decreased sensitivity of the cancer to a chemotherapy compared to previous sensitivity to the chemotherapy. Thus, chemotherapeutic resistance may occur despite the cancer previously responding to the chemotherapy. Chemotherapeutic resistance may refer to the ability of cancer cells to survive and grow despite chemotherapy treatment.

As used herein, the term “graft-versus-host disease” refers to a condition resulting from T cells from a tissue or organ transplant, i.e., a bone marrow transplant, react immunologically against the recipient's antigens by attacking recipient's cells and tissues.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease (e.g. cancer) or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. A “cancer subject” is a subject with cancer.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease (e.g. cancer), which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

As used herein, “sequential administration” includes that the administration of two agents (e.g., the compounds or compositions described herein) occurs separately on the same day or do not occur on a same day (e.g., occurs on consecutive days).

As used herein, “concurrent administration” includes overlapping in duration at least in part. For example, when two agents (e.g., any of the agents or class of agents described herein that has bioactivity) are administered concurrently, their administration occurs within a certain desired time. The agents administration may begin and end on the same day. The administration of one agent can also precede the administration of a second agent by day(s) as long as both agents are taken on the same day at least once. Similarly, the administration of one agent can extend beyond the administration of a second agent as long as both agents are taken on the same day at least once. The bioactive agents/agents do not have to be taken at the same time each day to include concurrent administration.

As used herein, “intermittent administration includes the administration of an agent for a period of time (which can be considered a “first period of administration”), followed by a time during which the agent is not taken or is taken at a lower maintenance dose (which can be considered “off-period”) followed by a period during which the agent is administered again (which can be considered a “second period of administration”). Generally, during the second phase of administration, the dosage level of the agent will match that administered during the first period of administration but can be increased or decreased as medically necessary.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

“Selective” or “selectivity” or the like of a compound refers to the compound's ability to discriminate between molecular targets.

“Specific”, “specifically”, “specificity”, or the like of a compound refers to the compound's ability to cause a particular action, such as inhibition, to a particular molecular target with minimal or no action to other proteins in the cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound or composition as disclosed herein, including embodiments thereof. Accordingly, an immune response can be activated or deactivated as a consequence of administration of an agent, e.g., a compound or composition as disclosed herein, including embodiments thereof.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

A “memory T cell” is a T cell that has previously encountered and responded to its cognate antigen during prior infection, encounter with cancer or previous vaccination. At a second encounter with its cognate antigen memory T cells can reproduce (divide) to mount a faster and stronger immune response than the first time the immune system responded to the pathogen.

A “regulatory T cell” or “suppressor T cell” is a lymphocyte which modulates the immune system, maintains tolerance to self-antigens, and prevents autoimmune disease.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with or without carriers.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection. Dosage forms may include sachets, capsules, chewable gels, granules, powders, tablets, wafers, or the like.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

Clostridium butyricum is an anaerobic endospore-forming Gram-positive butyric acid-producing bacillus. Clostridium butyricum can be found in human and animal gastrointestinal tracts. Clostridium butyricum MIYAIRI 588, also known as CBM or CBM588, is a strain of the bacteria Clostridium butyricum. Clostridium butyricum MIYAIRI 588 may have immunomodulatory, anti-inflammatory and antineoplastic properties. Clostridium butyricum MIYAIRI 588 may restore gut microbiota, and therefore may normalize intestinal immune responses.

Bifidobacterium is a genus of gram-positive anaerobic bacteria. Bifidobacterium may reside in the gastrointestinal tract, vagina and mouth (B. dentium) of mammals, including humans. Bifidobacteria are one of the major genera of bacteria that make up the gastrointestinal tract microbiota in mammals. In embodiments, strains of Bifidobacterium may be included in live biotherapeutic products. In embodiments, Bifidobacterium may normalize intestinal immune responses when used by itself or in combination with other bacteria as a live biotherapeutic product.

Dorea is a Gram-positive and non-spore-forming bacterial genus from the family Lachnospiraceae, and may be found in mammalian guts and may occur in which occur in human feces. Elevated levels of Dorea may be found in individuals with autoimmune conditions. Dorea may normalize intestinal immune responses when used by itself or in combination with other bacteria as a live biotherapeutic product.

Blautia is a gut microbial genus that is commonly found in the mammalian gut. Blautia produces butyric acid, which is used for cell processes throughout the body. Butyric acid can be used as a therapy for Irritable Bowel Syndrome (IBS); therefore Blautia may be a target for treating IBS. Blautia may have anti-inflammatory properties and may make the gut environment less tolerable to pathogenic bacteria. In embodiments, Blautia may be used by itself or in combination with other bacteria as a live biotherapeutic product.

Akkermansia municiphila is a species of human intestinal mucin-degrading bacterium. Akkermansia municiphila may be a target for treatment of obesity, diabetes, and inflammation. In embodiments, Akkermansia municiphila may be used by itself or in combination with other bacteria as a live biotherapeutic product.

A “live biotherapeutic product” or “LBP”, as used herein, refers to a biological product that contains live organisms and may be used for the prevention, treatment, or cure of a disease. For example, the live biotherapeutic product may contain microorganisms including live bacteria or yeast. The microorganisms may be naturally occurring, recombinant, or clonally selected. The live biotherapeutic product may be dried and remain alive for an extended period of time (e.g. 1-2 years). In embodiments, the live biotherapeutic product is a bacteria. In embodiments, the live biotherapeutic product is Clostridium butyricum Miyairi 588 (CBM588). In embodiments, the live biotherapeutic product is CBM588 in combination with another bacterial species, including but not limited to another strain of Clostridium butyricum, a strain from the genus Bifidobacterium, a strain from the genus Dorea, a strain from the genus Blautia, or Akkermansia municiphila. In embodiments, the live biotherapeutic product is CBM588 in combination with multiple other bacterial species, including but not limited to one or more other strains of Clostridium butyricum, one or more strains from the genus Bifidobacterium, one or more strains from the genus Dorea, one or more strains from the genus Blautia, or Akkermansia municiphila.

As used herein, a “recombinant live biotherapeutic product” or “recombinant LBP” refers to a live biotherapeutic product including microorganisms that have been genetically modified through the purposeful addition, deletion, or modification of genetic material.

The term “Clostridium butyricum MIYAIRI 588 live biotherapeutic product” or “CBM588 LBP” as used herein refers to Clostridium butyricum MIYAIRI 588 when used for the prevention, treatment, or cure of a disease. CBM588 is described in further detail in Japanese patent JPH1142081A (“Production of Clostridium butyricum”, published Feb. 16, 1999), which is incorporated herein by reference in its entirety for all purposes. CBM588 was deposited with the Ministry of International Trade and Industry Agency of Industrial Science Research Institute under the accession number of FERM BP-2789 (FERM BP-2789). In embodiments, CBM388 LBP is administered in combination with another bacterial species, including but not limited to another strain of Clostridium butyricum, a strain from the genus Bifidobacterium, a strain from the genus Dorea, a strain from the genus Blautia, or Akkermansia municiphila. In embodiments, CBM388 LBP is administered in combination with multiple other bacterial species, including but not limited to one or more other strains of Clostridium butyricum, one or more strains from the genus Bifidobacterium, one or more strains from the genus Dorea, one or more strains from the genus Blautia, and/or Akkermansia municiphila.

As used herein, the term “hematopoietic stem cell transplantation” refers to transplantation of multipotent hematopoietic stem cells, usually derived from bone marrow, peripheral blood, or umbilical cord blood.

As used herein, the term “allogeneic hematopoietic stem cell transplantation” refers to transplantation of multipotent hematopoietic stem cells, in which the healthy stem cells come from the blood or bone marrow of a related donor who is not an identical twin of the patient or from an unrelated donor who is genetically similar to the patient.

As used herein, the term “gut microbiota” refers to the microorganisms including bacteria, archaea and microscopic eukaryotes that live in the digestive tracts of humans and other animals.

As used herein, the term “sickle cell disease” refers to an inherited red blood cell disorder characterized by red blood cells of a changed shape, e.g., crescent moon-shaped sickle shape. These sickle red blood cells are stiff and sticky, so they can block the blood flow or stick to the blood vessel walls, and break down inside the blood vessels.

As used herein, the term “autoimmine disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

### Methods

Without wishing to be bound by scientific theory, Clostridium butyricum (e.g. Clostridium butyricum MIYAIRI 588 live biotherapeutic product (CBM588 LBP)) is contemplated to modulate levels of specific types of bacteria (e.g. bifidobacterium) in the gastrointestinal tract microbiome. In embodiments, increases or decreases in levels of specific types of bacteria enhance the chances for treating graft-versus-host disease in a subject undergoing allogeneic hematopoietic stem cell transplantation.

Provided herein is a method for treating graft-versus-host disease in a subject in need thereof, the method comprising administering to the subject a therapeutically effective amount of Clostridium butyricum.

In embodiments, provided is a method for treating graft-versus-host disease in a subject in need thereof, the method comprising administering to the subject an effective amount of Clostridium butyricum live biotherapeutic product (LBP).

In embodiments, provided is a method for treating graft-versus-host disease in a subject in need thereof, the method comprising administering to the subject an effective amount of Clostridium butyricum MIYAIRI 588 live biotherapeutic product (CBM588 LBP).

In embodiments, the subject is a subject who is undergoing allogeneic hematopoietic stem cell transplantation. In embodiments, the subject is a subject that has undergone allogeneic hematopoietic stem cell transplantation.

In embodiments, the subject who is undergoing allogeneic hematopoietic stem cell transplantation is a cancer subject. In embodiments, the subject that has undergone allogeneic hematopoietic stem cell transplantation is a cancer subject. In embodiments, the subject who is undergoing allogeneic hematopoietic stem cell transplantation is a hematological cancer subject. In embodiments, the subject that has undergone allogeneic hematopoietic stem cell transplantation is a hematological cancer subject.

In embodiments, the hematologic cancer is leukemia, lymphoma, myeloma or myelodysplastic syndrome. In embodiments, the hematologic cancer is leukemia. In embodiments, the hematologic cancer is lymphoma. In embodiments, the hematologic cancer is myeloma. In embodiments, the hematologic cancer is myelodysplastic syndrome.

In embodiments, the subject who is undergoing allogeneic hematopoietic stem cell transplantation suffers from non-cancer hematological disorder. In embodiments, the subject that has undergone allogeneic hematopoietic stem cell transplantation suffers from non-cancer hematological disorder. In embodiments, the subject who is undergoing allogeneic hematopoietic stem cell transplantation has non-cancer hematological disorder. In embodiments, the subject that has undergone allogeneic hematopoietic stem cell transplantation has non-cancer hematological disorder. In embodiments, the non-cancer hematological disorder is a sickle cell disorder.

In embodiments, the subject who is undergoing allogeneic hematopoietic stem cell transplantation suffers from an autoimmune disease. In embodiments, the subject that has undergone allogeneic hematopoietic stem cell transplantation suffers from an autoimmune disease. In embodiments, the subject who is undergoing allogeneic hematopoietic stem cell transplantation has an autoimmune disease. In embodiments, the subject that has undergone allogeneic hematopoietic stem cell transplantation has an autoimmune disease.

In embodiments, the CBM588 LBP is administered in combination with another strain of Clostridium butyricum, a strain from the genus Bifidobacterium, a strain from the genus Dorea, a strain from the genus Blautia, or Akkermansia municiphila. In embodiments, the CBM588 LBP is administered with another strain of Clostridium butyricum. In embodiments, the CBM588 LBP is administered with a strain from the genus Bifidobacterium. In embodiments, the CBM588 LBP is administered with a strain from the genus Dorea. In embodiments, the CBM588 LBP is administered with a strain from the genus Blautia. In embodiments, the CBM588 LBP is administered with Akkermansia municiphila. In embodiments the CBM388 LBP is administered in combination with of one or more of other bacterial species, including but not limited to one or more other strains of Clostridium butyricum, one or more strains from the genus Bifidobacterium, one or more strains from the genus Dorea, one or more strains from the genus Blautia, and/or Akkermansia municiphila.

In embodiments, provided is a method for treating graft-versus-host disease in a subject in need thereof, the method comprising administering to the subject the CBM588 LBP in a dose of about 50 mg per day to about 250 mg per day.

In embodiments, the dose of CBM588 LBP is from about 50 mg per day to about 250 mg per day. In embodiments, the dose of CBM588 LBP is from about 75 mg per day to about 225 mg per day. In embodiments, the dose of CBM588 LBP is from about 100 mg per day to about 200 mg per day. In embodiments, the dose of CBM588 LBP is from about 125 mg per day to about 175 mg per day.

In embodiments, the dose of CBM588 LBP is about 50 mg per day. In embodiments, the dose of CBM588 LBP is about 60 mg per day. In embodiments, the dose of CBM588 LBP is about 70 mg per day. In embodiments, the dose of CBM588 LBP is about 80 mg per day. In embodiments, the dose of CBM588 LBP is about 90 mg per day. In embodiments, the dose of CBM588 LBP is about 100 mg per day. In embodiments, the dose of CBM588 LBP is about 110 mg per day. In embodiments, the dose of CBM588 LBP is about 120 mg per day. In embodiments, the dose of CBM588 LBP is about 130 mg per day. In embodiments, the dose of CBM588 LBP is about 140 mg per day. In embodiments, the dose of CBM588 LBP is about 150 mg per day. In embodiments, the dose of f CBM588 LBP is about 160 mg per day. In embodiments, the dose of CBM588 LBP is about 170 mg per day. In embodiments, the dose of CBM588 LBP is about 180 mg per day. In embodiments, the dose of CBM588 LBP is about 190 mg per day. In embodiments, the dose of CBM588 LBP is about 200 mg per day. In embodiments, the dose of CBM588 LBP is about 225 mg per day. In embodiments, the dose of CBM588 LBP is about 250 mg per day.

In embodiments, provided is a method for treating graft-versus-host disease in a subject in need thereof, the method comprising administering the CBM588 LBP to the subject in the dose of about 60 mg per day. In embodiments, provided is a method for treating graft-versus-host disease in a subject in need thereof, the method comprising administering the CBM588 LBP to the subject in the dose of about 80 mg per day. In embodiments, provided is a method for treating graft-versus-host disease in a subject in need thereof, the method comprising administering the CBM588 LBP to the subject in the dose of about 100 mg per day. In embodiments, provided is a method for treating graft-versus-host disease in a subject in need thereof, the method comprising administering the CBM588 LBP to the subject in the dose of about 120 mg per day. In embodiments, provided is a method for treating graft-versus-host disease in a subject in need thereof, the method comprising administering the CBM588 LBP to the subject in the dose of about 140 mg per day. In embodiments, provided is a method for treating graft-versus-host disease in a subject in need thereof, the method comprising administering the CBM588 LBP to the subject in the dose of about 160 mg per day. In embodiments, provided is a method for treating graft-versus-host disease in a subject in need thereof, the method comprising administering the CBM588 LBP to the subject in the dose of about 180 mg per day.

In embodiments, provided is a method for treating graft-versus-host disease in a subject in need thereof, the method comprising administering the CBM588 LBP to the subject once a day. In embodiments, provided is a method for treating graft-versus-host disease in a subject in need thereof, the method comprising administering the CBM588 LBP to the subject twice a day. In embodiments, provided is a method for treating graft-versus-host disease in a subject in need thereof, the method comprising administering the CBM588 LBP to the subject three times a day.

In embodiments, provided is a method for treating graft-versus-host disease in a subject in need thereof, the method comprising administering the CBM588 LBP orally. In embodiments, provided is a method for treating graft-versus-host disease in a subject in need thereof, the method comprising administering the CBM588 LBP parentherally.

In embodiments, provided is a method for treating graft-versus-host disease in a subject in need thereof, the method comprising administering the CBM588 LBP to the subject starting from day −8 of the hematopoietic stem cell transplantation to day +28. In embodiments, provided is a method for treating graft-versus-host disease in a subject in need thereof, the method comprising administering the CBM588 LBP to the subject starting from day −7 of the hematopoietic stem cell transplantation to day +28. In embodiments, provided is a method for treating graft-versus-host disease in a subject in need thereof, the method comprising administering the CBM588 LBP to the subject starting from day −6 of the hematopoietic stem cell transplantation to day +28. In embodiments, provided is a method for treating graft-versus-host disease in a subject in need thereof, the method comprising administering the CBM588 LBP to the subject starting from day −5 of the hematopoietic stem cell transplantation to day +28. In embodiments, provided is a method for treating graft-versus-host disease in a subject in need thereof, the method comprising administering the CBM588 LBP to the subject starting from day −4 of the hematopoietic stem cell transplantation to day +28. In embodiments, provided is a method for treating graft-versus-host disease in a subject in need thereof, the method comprising administering the CBM588 LBP to the subject starting from day −3 of the hematopoietic stem cell transplantation to day +28. In embodiments, provided is a method for treating graft-versus-host disease in a subject in need thereof, the method comprising administering the CBM588 LBP to the subject starting from day −2 of the hematopoietic stem cell transplantation to day +28. In embodiments, provided is a method for treating graft-versus-host disease in a subject in need thereof, the method comprising administering the CBM588 LBP to the subject starting from day −1 of the hematopoietic stem cell transplantation to day +28. In embodiments, provided is a method for treating graft-versus-host disease in a subject in need thereof, the method comprising administering the CBM588 LBP to the subject starting simultaneously with the hematopoietic stem cell transplantation to day +28.

In embodiments, provided is a method for treating graft-versus-host disease in a subject in need thereof, the method comprising administering the CBM588 LBP to the subject starting from day −8 of the hematopoietic stem cell transplantation to the day of discharge from the hospital. In embodiments, provided is a method for treating graft-versus-host disease in a subject in need thereof, the method comprising administering the CBM588 LBP to the subject starting from day −7 of the hematopoietic stem cell transplantation to the day of discharge from the hospital. In embodiments, provided is a method for treating graft-versus-host disease in a subject in need thereof, the method comprising administering the CBM588 LBP to the subject starting from day −6 of the hematopoietic stem cell transplantation to the day of discharge from the hospital. In embodiments, provided is a method for treating graft-versus-host disease in a subject in need thereof, the method comprising administering the CBM588 LBP to the subject starting from day −5 of the hematopoietic stem cell transplantation to the day of discharge from the hospital. In embodiments, provided is a method for treating graft-versus-host disease in a subject in need thereof, the method comprising administering the CBM588 LBP to the subject starting from day −4 of the hematopoietic stem cell transplantation to the day of discharge from the hospital. In embodiments, provided is a method for treating graft-versus-host disease in a subject in need thereof, the method comprising administering the CBM588 LBP to the subject starting from day −3 of the hematopoietic stem cell transplantation to the day of discharge from the hospital. In embodiments, provided is a method for treating graft-versus-host disease in a subject in need thereof, the method comprising administering the CBM588 LBP to the subject starting from day −2 of the hematopoietic stem cell transplantation to the day of discharge from the hospital. In embodiments, provided is a method for treating graft-versus-host disease in a subject in need thereof, the method comprising administering the CBM588 LBP to the subject starting from day −1 of the hematopoietic stem cell transplantation to the day of discharge from the hospital. In embodiments, provided is a method for treating graft-versus-host disease in a subject in need thereof, the method comprising administering the CBM588 LBP to the subject starting simultaneously with the hematopoietic stem cell transplantation to the day of discharge from the hospital.

In embodiments, provided is a method for treating graft-versus-host disease in a subject in need thereof, the method comprising administering the CBM588 LBP to the subject for about 1 week to about 100 weeks. In embodiments, CBM588 LBP may be administered for about 10 weeks to about 100 weeks. In embodiments, CBM588 LBP may be administered for about 20 weeks to about 100 weeks. In embodiments, CBM588 LBP may be administered for about 30 weeks to about 100 weeks. In embodiments, CBM588 LBP may be administered for about 40 weeks to about 100 weeks. In embodiments, CBM588 LBP may be administered for about 50 weeks to about 100 weeks. In embodiments, CBM588 LBP may be administered for about 60 weeks to about 100 weeks. In embodiments, CBM588 LBP may be administered for about 70 weeks to about 100 weeks. In embodiments, CBM588 LBP may be administered for about 80 weeks to about 100 weeks. In embodiments, CBM588 LBP may be administered for about 90 weeks to about 100 weeks.

Provided herein is a method of increasing gut biodiversity in a subject in need thereof, the method comprising administering to the subject a therapeutically effective amount of Clostridium butyricum.

In embodiments, provided is a method of increasing gut biodiversity in a subject in need thereof, the method comprising administering to the subject an effective amount of Clostridium butyricum live biotherapeutic product (LBP).

In embodiments, provided is a method of increasing gut biodiversity in a subject in need thereof, the method comprising administering to the subject an effective amount of Clostridium butyricum MIYAIRI 588 live biotherapeutic product (CBM588 LBP).

In embodiments, the subject is a subject who is undergoing allogeneic hematopoietic stem cell transplantation. In embodiments, the subject is a subject that has undergone allogeneic hematopoietic stem cell transplantation.

In embodiments, the subject who is undergoing allogeneic hematopoietic stem cell transplantation is a cancer subject. In embodiments, the subject that has undergone allogeneic hematopoietic stem cell transplantation is a cancer subject. In embodiments, the subject who is undergoing allogeneic hematopoietic stem cell transplantation is a hematological cancer subject. In embodiments, the subject that has undergone allogeneic hematopoietic stem cell transplantation is a hematological cancer subject.

In embodiments, the hematologic cancer is leukemia, lymphoma, myeloma or myelodysplastic syndrome. In embodiments, the hematologic cancer is leukemia. In embodiments, the hematologic cancer is lymphoma. In embodiments, the hematologic cancer is myeloma. In embodiments, the hematologic cancer is myelodysplastic syndrome.

In embodiments, the subject who is undergoing allogeneic hematopoietic stem cell transplantation suffers from non-cancer hematological disorder. In embodiments, the subject who is undergoing allogeneic hematopoietic stem cell transplantation has a non-cancer hematological disorder. In embodiments, the subject that has undergone allogeneic hematopoietic stem cell transplantation suffers from non-cancer hematological disorder. In embodiments, the subject that has undergone allogeneic hematopoietic stem cell transplantation has a non-cancer hematological disorder. In embodiments, non-cancer hematological disorder is a sickle cell disorder.

In embodiments, the subject who is undergoing allogeneic hematopoietic stem cell transplantation suffers from an autoimmune disease. In embodiments, the subject who is undergoing allogeneic hematopoietic stem cell transplantation has an autoimmune disease. In embodiments, the subject that has undergone allogeneic hematopoietic stem cell transplantation suffers from an autoimmune disease. In embodiments, the subject that has undergone allogeneic hematopoietic stem cell transplantation has an autoimmune disease.

In embodiments, the CBM588 LBP is administered in combination with another strain of Clostridium butyricum, a strain from the genus Bifidobacterium, a strain from the genus Dorea, a strain from the genus Blautia, or Akkermansia municiphila. In embodiments, the CBM588 LBP is administered with another strain of Clostridium butyricum. In embodiments, the CBM588 LBP is administered with a strain from the genus Bifidobacterium. In embodiments, the CBM588 LBP is administered with a strain from the genus Dorea. In embodiments, the CBM588 LBP is administered with a strain from the genus Blautia. In embodiments, the CBM588 LBP is administered with Akkermansia municiphila. In embodiments the CBM388 LBP is administered in combination with of one or more of other bacterial species, including but not limited to one or more other strains of Clostridium butyricum, one or more strains from the genus Bifidobacterium, one or more strains from the genus Dorea, one or more strains from the genus Blautia, and/or Akkermansia municiphila.

In embodiments, provided is a method of increasing gut biodiversity in a subject in need thereof, the method comprising administering to the subject the CBM588 LBP in a dose of about 50 mg per day to about 250 mg per day.

In embodiments, provided is a method of increasing gut biodiversity in a subject in need thereof, the method comprising administering the CBM588 LBP to the subject in the dose of about 60 mg per day. In embodiments, provided is a method of increasing gut biodiversity in a subject in need thereof, the method comprising administering the CBM588 LBP to the subject in the dose of about 80 mg per day. In embodiments, provided is a method of increasing gut biodiversity in a subject in need thereof, the method comprising administering the CBM588 LBP to the subject in the dose of about 100 mg per day. In embodiments, provided is a method of increasing gut biodiversity in a subject in need thereof, the method comprising administering the CBM588 LBP to the subject in the dose of about 120 mg per day. In embodiments, provided is a method of increasing gut biodiversity in a subject in need thereof, the method comprising administering the CBM588 LBP to the subject in the dose of about 140 mg per day. In embodiments, provided is a method of increasing gut biodiversity in a subject in need thereof, the method comprising administering the CBM588 LBP to the subject in the dose of about 160 mg per day. In embodiments, provided is a method of increasing gut biodiversity in a subject in need thereof, the method comprising administering the CBM588 LBP to the subject in the dose of about 180 mg per day.

In embodiments, provided is a method of increasing gut biodiversity in a subject in need thereof, the method comprising administering the CBM588 LBP to the subject once a day. In embodiments, provided is a method of increasing gut biodiversity in a subject in need thereof, the method comprising administering the CBM588 LBP to the subject twice a day. In embodiments, provided is a method of increasing gut biodiversity in a subject in need thereof, the method comprising administering the CBM588 LBP to the subject three times a day.

In embodiments, provided is a method of increasing gut biodiversity in a subject in need thereof, the method comprising administering the CBM588 LBP orally. In embodiments, provided is a method of increasing gut biodiversity in a subject in need thereof, the method comprising administering the CBM588 LBP parentherally.

In embodiments, provided is a method of increasing gut biodiversity in a subject in need thereof, the method comprising administering the CBM588 LBP to the subject starting from day −8 of the hematopoietic stem cell transplantation to day +28. In embodiments, provided is a method of increasing gut biodiversity in a subject in need thereof, the method comprising administering the CBM588 LBP to the subject starting from day −7 of the hematopoietic stem cell transplantation to day +28. In embodiments, provided is a method of increasing gut biodiversity in a subject in need thereof, the method comprising administering the CBM588 LBP to the subject starting from day −6 of the hematopoietic stem cell transplantation to day +28. In embodiments, provided is a method of increasing gut biodiversity in a subject in need thereof, the method comprising administering the CBM588 LBP to the subject starting from day −5 of the hematopoietic stem cell transplantation to day +28. In embodiments, provided is a method of increasing gut biodiversity in a subject in need thereof, the method comprising administering the CBM588 LBP to the subject starting from day −4 of the hematopoietic stem cell transplantation to day +28. In embodiments, provided is a method of increasing gut biodiversity in a subject in need thereof, the method comprising administering the CBM588 LBP to the subject starting from day −3 of the hematopoietic stem cell transplantation to day +28. In embodiments, provided is a method of increasing gut biodiversity in a subject in need thereof, the method comprising administering the CBM588 LBP to the subject starting from day −2 of the hematopoietic stem cell transplantation to day +28. In embodiments, provided is a method of increasing gut biodiversity in a subject in need thereof, the method comprising administering the CBM588 LBP to the subject starting from day −1 of the hematopoietic stem cell transplantation to day +28. In embodiments, provided is a method of increasing gut biodiversity in a subject in need thereof, the method comprising administering the CBM588 LBP to the subject starting simultaneously with the hematopoietic stem cell transplantation to day +28.

In embodiments, provided is a method of increasing gut biodiversity in a subject in need thereof, the method comprising administering the CBM588 LBP to the subject starting from day −8 of the hematopoietic stem cell transplantation to the day of discharge from the hospital. In embodiments, provided is a method of increasing gut biodiversity in a subject in need thereof, the method comprising administering the CBM588 LBP to the subject starting from day −7 of the hematopoietic stem cell transplantation to the day of discharge from the hospital. In embodiments, provided is a method of increasing gut biodiversity in a subject in need thereof, the method comprising administering the CBM588 LBP to the subject starting from day −6 of the hematopoietic stem cell transplantation to the day of discharge from the hospital. In embodiments, provided is a method of increasing gut biodiversity in a subject in need thereof, the method comprising administering the CBM588 LBP to the subject starting from day −5 of the hematopoietic stem cell transplantation to the day of discharge from the hospital. In embodiments, provided is a method of increasing gut biodiversity in a subject in need thereof, the method comprising administering the CBM588 LBP to the subject starting from day −4 of the hematopoietic stem cell transplantation to the day of discharge from the hospital. In embodiments, provided is a method of increasing gut biodiversity in a subject in need thereof, the method comprising administering the CBM588 LBP to the subject starting from day −3 of the hematopoietic stem cell transplantation to the day of discharge from the hospital. In embodiments, provided is a method of increasing gut biodiversity in a subject in need thereof, the method comprising administering the CBM588 LBP to the subject starting from day −2 of the hematopoietic stem cell transplantation to the day of discharge from the hospital. In embodiments, provided is a method of increasing gut biodiversity in a subject in need thereof, the method comprising administering the CBM588 LBP to the subject starting from day −1 of the hematopoietic stem cell transplantation to the day of discharge from the hospital. In embodiments, provided is a method of increasing gut biodiversity in a subject in need thereof, the method comprising administering the CBM588 LBP to the subject starting simultaneously with the hematopoietic stem cell transplantation to the day of discharge from the hospital.

In embodiments, provided is a method for increasing gut biodiversity in a subject in need thereof, the method comprising administering the CBM588 LBP to the subject for about 1 week to about 100 weeks. In embodiments, CBM588 LBP may be administered for about 10 weeks to about 100 weeks. In embodiments, CBM588 LBP may be administered for about 20 weeks to about 100 weeks. In embodiments, CBM588 LBP may be administered for about 30 weeks to about 100 weeks. In embodiments, CBM588 LBP may be administered for about 40 weeks to about 100 weeks. In embodiments, CBM588 LBP may be administered for about 50 weeks to about 100 weeks. In embodiments, CBM588 LBP may be administered for about 60 weeks to about 100 weeks. In embodiments, CBM588 LBP may be administered for about 70 weeks to about 100 weeks. In embodiments, CBM588 LBP may be administered for about 80 weeks to about 100 weeks. In embodiments, CBM588 LBP may be administered for about 90 weeks to about 100 weeks.

Provided herein is a method of improving clinical outcome of a patient undergoing allogeneic hematopoietic stem cell transplantation, said method comprising administering to said patient a therapeutically effective amount of Clostridium butyricum.

In embodiments, provided is a method of improving clinical outcome of a patient undergoing allogeneic hematopoietic stem cell transplantation, said method comprising administering to said patient a therapeutically effective amount of Clostridium butyricum live biotherapeutic product (LBP).

In embodiments, provided is a method of improving clinical outcome of a patient undergoing allogeneic hematopoietic stem cell transplantation, said method comprising administering to said patient a therapeutically effective amount of Clostridium butyricum MIYAIRI 588 live biotherapeutic product (CBM588 LBP).

In embodiments, the subject is a subject who is undergoing allogeneic hematopoietic stem cell transplantation. In embodiments, the subject is a subject that has undergone allogeneic hematopoietic stem cell transplantation.

In embodiments, the subject who is undergoing allogeneic hematopoietic stem cell transplantation is a cancer subject. In embodiments, the subject that has undergone allogeneic hematopoietic stem cell transplantation is a cancer subject. In embodiments, the subject who is undergoing allogeneic hematopoietic stem cell transplantation is a hematological cancer subject. In embodiments, the subject that has undergone allogeneic hematopoietic stem cell transplantation is a hematological cancer subject.

In embodiments, the hematologic cancer is leukemia, lymphoma, myeloma or myelodysplastic syndrome. In embodiments, the hematologic cancer is leukemia. In embodiments, the hematologic cancer is lymphoma. In embodiments, the hematologic cancer is myeloma. In embodiments, the hematologic cancer is myelodysplastic syndrome.

In embodiments, the subject who is undergoing allogeneic hematopoietic stem cell transplantation suffers from non-cancer hematological disorder. In embodiments, the subject who is undergoing allogeneic hematopoietic stem cell transplantation has a non-cancer hematological disorder. In embodiments, the subject that has undergone allogeneic hematopoietic stem cell transplantation suffers from non-cancer hematological disorder. In embodiments, the subject that has undergone allogeneic hematopoietic stem cell transplantation has a non-cancer hematological disorder. In embodiments, non-cancer hematological disorder is a sickle cell disorder.

In embodiments, the subject who is undergoing allogeneic hematopoietic stem cell transplantation suffers from an autoimmune disease. In embodiments, the subject who is undergoing allogeneic hematopoietic stem cell transplantation has an autoimmune disease. In embodiments, the subject that has undergone allogeneic hematopoietic stem cell transplantation suffers from an autoimmune disease. In embodiments, the subject that has undergone allogeneic hematopoietic stem cell transplantation has an autoimmune disease.

In embodiments, the CBM588 LBP is administered in combination with another strain of Clostridium butyricum, a strain from the genus Bifidobacterium, a strain from the genus Dorea, a strain from the genus Blautia, or Akkermansia municiphila. In embodiments, the CBM588 LBP is administered with another strain of Clostridium butyricum. In embodiments, the CBM588 LBP is administered with a strain from the genus Bifidobacterium. In embodiments, the CBM588 LBP is administered with a strain from the genus Dorea. In embodiments, the CBM588 LBP is administered with a strain from the genus Blautia. In embodiments, the CBM588 LBP is administered with Akkermansia municiphila. In embodiments the CBM388 LBP is administered in combination with of one or more of other bacterial species, including but not limited to one or more other strains of Clostridium butyricum, one or more strains from the genus Bifidobacterium, one or more strains from the genus Dorea, one or more strains from the genus Blautia, and/or Akkermansia municiphila.

In embodiments, provided is a method of improving clinical outcome in a subject in need thereof, the method comprising administering to the subject the CBM588 LBP in a dose of about 50 mg per day to about 250 mg per day.

In embodiments, provided is a method of improving clinical outcome in a subject in need thereof, the method comprising administering the CBM588 LBP to the subject in the dose of about 60 mg per day. In embodiments, provided is a method of improving clinical outcome in a subject in need thereof, the method comprising administering the CBM588 LBP to the subject in the dose of about 80 mg per day. In embodiments, provided is a method of improving clinical outcome in a subject in need thereof, the method comprising administering the CBM588 LBP to the subject in the dose of about 100 mg per day. In embodiments, provided is a method of improving clinical outcome in a subject in need thereof, the method comprising administering the CBM588 LBP to the subject in the dose of about 120 mg per day. In embodiments, provided is a method of improving clinical outcome in a subject in need thereof, the method comprising administering the CBM588 LBP to the subject in the dose of about 140 mg per day. In embodiments, provided is a method of improving clinical outcome in a subject in need thereof, the method comprising administering the CBM588 LBP to the subject in the dose of about 160 mg per day. In embodiments, provided is a method of improving clinical outcome in a subject in need thereof, the method comprising administering the CBM588 LBP to the subject in the dose of about 180 mg per day.

In embodiments, provided is a method of improving clinical outcome in a subject in need thereof, the method comprising administering the CBM588 LBP to the subject once a day. In embodiments, provided is a method of improving clinical outcome in a subject in need thereof, the method comprising administering the CBM588 LBP to the subject twice a day. In embodiments, provided is a method of improving clinical outcome in a subject in need thereof, the method comprising administering the CBM588 LBP to the subject three times a day.

In embodiments, provided is a method of improving clinical outcome in a subject in need thereof, the method comprising administering the CBM588 LBP orally. In embodiments, provided is a method of improving clinical outcome in a subject in need thereof, the method comprising administering the CBM588 LBP parentherally.

In embodiments, provided is a method of improving clinical outcome in a subject in need thereof, the method comprising administering the CBM588 LBP to the subject starting from day −8 of the hematopoietic stem cell transplantation to day +28. In embodiments, provided is a method of improving clinical outcome in a subject in need thereof, the method comprising administering the CBM588 LBP to the subject starting from day −7 of the hematopoietic stem cell transplantation to day +28. In embodiments, provided is a method of improving clinical outcome in a subject in need thereof, the method comprising administering the CBM588 LBP to the subject starting from day −6 of the hematopoietic stem cell transplantation to day +28. In embodiments, provided is a method of improving clinical outcome in a subject in need thereof, the method comprising administering the CBM588 LBP to the subject starting from day −5 of the hematopoietic stem cell transplantation to day +28. In embodiments, provided is a method of improving clinical outcome in a subject in need thereof, the method comprising administering the CBM588 LBP to the subject starting from day −4 of the hematopoietic stem cell transplantation to day +28. In embodiments, provided is a method of improving clinical outcome in a subject in need thereof, the method comprising administering the CBM588 LBP to the subject starting from day −3 of the hematopoietic stem cell transplantation to day +28. In embodiments, provided is a method of improving clinical outcome in a subject in need thereof, the method comprising administering the CBM588 LBP to the subject starting from day −2 of the hematopoietic stem cell transplantation to day +28. In embodiments, provided is a method of improving clinical outcome in a subject in need thereof, the method comprising administering the CBM588 LBP to the subject starting from day −1 of the hematopoietic stem cell transplantation to day +28. In embodiments, provided is a method of improving clinical outcome in a subject in need thereof, the method comprising administering the CBM588 LBP to the subject starting simultaneously with the hematopoietic stem cell transplantation to day +28.

In embodiments, provided is a method of improving clinical outcome in a subject in need thereof, the method comprising administering the CBM588 LBP to the subject starting from day −8 of the hematopoietic stem cell transplantation to the day of discharge from the hospital. In embodiments, provided is a method of improving clinical outcome in a subject in need thereof, the method comprising administering the CBM588 LBP to the subject starting from day −7 of the hematopoietic stem cell transplantation to the day of discharge from the hospital. In embodiments, provided is a method of improving clinical outcome in a subject in need thereof, the method comprising administering the CBM588 LBP to the subject starting from day −6 of the hematopoietic stem cell transplantation to the day of discharge from the hospital. In embodiments, provided is a method of improving clinical outcome in a subject in need thereof, the method comprising administering the CBM588 LBP to the subject starting from day −5 of the hematopoietic stem cell transplantation to the day of discharge from the hospital. In embodiments, provided is a method of improving clinical outcome in a subject in need thereof, the method comprising administering the CBM588 LBP to the subject starting from day −4 of the hematopoietic stem cell transplantation to the day of discharge from the hospital. In embodiments, provided is a method of improving clinical outcome in a subject in need thereof, the method comprising administering the CBM588 LBP to the subject starting from day −3 of the hematopoietic stem cell transplantation to the day of discharge from the hospital. In embodiments, provided is a method of improving clinical outcome in a subject in need thereof, the method comprising administering the CBM588 LBP to the subject starting from day −2 of the hematopoietic stem cell transplantation to the day of discharge from the hospital. In embodiments, provided is a method of improving clinical outcome in a subject in need thereof, the method comprising administering the CBM588 LBP to the subject starting from day −1 of the hematopoietic stem cell transplantation to the day of discharge from the hospital. In embodiments, provided is a method of improving clinical outcome in a subject in need thereof, the method comprising administering the CBM588 LBP to the subject starting simultaneously with the hematopoietic stem cell transplantation to the day of discharge from the hospital.

In embodiments, provided is a method of improving clinical outcome in a subject in need thereof, the method comprising administering the CBM588 LBP to the subject for about 1 week to about 100 weeks. In embodiments, CBM588 LBP may be administered for about 10 weeks to about 100 weeks. In embodiments, CBM588 LBP may be administered for about 20 weeks to about 100 weeks. In embodiments, CBM588 LBP may be administered for about 30 weeks to about 100 weeks. In embodiments, CBM588 LBP may be administered for about 40 weeks to about 100 weeks. In embodiments, CBM588 LBP may be administered for about 50 weeks to about 100 weeks. In embodiments, CBM588 LBP may be administered for about 60 weeks to about 100 weeks. In embodiments, CBM588 LBP may be administered for about 70 weeks to about 100 weeks. In embodiments, CBM588 LBP may be administered for about 80 weeks to about 100 weeks. In embodiments, CBM588 LBP may be administered for about 90 weeks to about 100 weeks.

In embodiments, an improved clinical outcome is shown by increased microbiome diversity and improved immune-biologic endpoints. In embodiments, an improved clinical outcome is demonstrated by increased microbiome diversity. In embodiments, an improved clinical outcome is demonstrated by improved immune-biologic endpoints. In embodiments, the immune-biologic endpoints are exemplified by cytokines levels, cellular immune reconstitution, and metabolomics. In embodiments, the metabolomics are represented by short chain fatty acids. In embodiments, the short chain fatty acid is an acetate fatty acid, propionate fatty acid, butyrate fatty acid, and the like.

### Pharmaceutical Compositions

Applicants have discovered that the compositions described herein including embodiments thereof are surprisingly useful for treating graft-versus-host disease. In addition, the Clostridium butyricum described herein may enhance gut biodiversity and improve clinical outcome of a patient undergoing allogeneic hematopoietic stem cell transplantation.

Provided is a pharmaceutical composition comprising Clostridium butyricum. In embodiments, the Clostridium butyricum is a live biotherapeutic product. In embodiments, the Clostridium butyricum live biotherapeutic product is Clostridium butyricum MIYAIRI 588 live biotherapeutic product.

In embodiments, the pharmaceutical composition includes another strain of Clostridium butyricum, a strain from the genus Bifidobacterium, a strain from the genus Dorea, a strain from the genus Blautia, or Akkermansia municiphila. In embodiments, the pharmaceutical composition includes another strain of Clostridium butyricum. In embodiments, the pharmaceutical composition includes a strain from the genus Bifidobacterium. In embodiments, the pharmaceutical composition includes a strain from the genus Dorea. In embodiments, the pharmaceutical composition includes a strain from the genus Blautia. In embodiments, the pharmaceutical composition includes Akkermansia municiphila. In embodiments, the pharmaceutical composition includes CBM388 with of one or more of another bacterial species, including but not limited to one or more other strains of Clostridium butyricum, one or more strains from the genus Bifidobacterium, one or more strains from the genus Dorea, one or more strains from the genus Blautia, and/or Akkermansia municiphila.

In embodiments, the Clostridium butyricum is administered with a potency of about 0.1×10⁹ colony forming unit (CFU)/dose to about 10×10⁹ CFU/dose. In embodiments, the Clostridium butyricum is administered with a potency of about 0.5×10⁹ CFU/dose to about 10×10⁹ CFU/dose. In embodiments, the Clostridium butyricum is administered with a potency of about 1.0×10⁹ CFU/dose to about 10×10⁹ CFU/dose. In embodiments, the Clostridium butyricum is administered with a potency of about 1.5×10⁹ CFU/dose to about 10×10⁹ CFU/dose. In embodiments, the Clostridium butyricum is administered with a potency of about 2.0×10⁹ CFU/dose to about 10×10⁹ CFU/dose. In embodiments, the Clostridium butyricum is administered with a potency of about 2.5×10⁹ CFU/dose to about 10×10⁹ CFU/dose. In embodiments, the Clostridium butyricum is administered with a potency of about 3.0×10⁹ CFU/dose to about 10×10⁹ CFU/dose. In embodiments, the Clostridium butyricum is administered with a potency of about 3.5×10⁹ CFU/dose to about 10×10⁹ CFU/dose. In embodiments, the Clostridium butyricum is administered with a potency of about 4.0×10⁹ CFU/dose to about 10×10⁹ CFU/dose. In embodiments, the Clostridium butyricum is administered with a potency of about 4.5×10⁹ CFU/dose to about 10×10⁹ CFU/dose. In embodiments, the Clostridium butyricum is administered with a potency of about 5.0×10⁹ CFU/dose to about 10×10⁹ CFU/dose. In embodiments, the Clostridium butyricum is administered with a potency of about 5.5×10⁹ CFU/dose to about 10×10⁹ CFU/dose. In embodiments, the Clostridium butyricum is administered with a potency of about 6.0×10⁹ CFU/dose to about 10×10⁹ CFU/dose. In embodiments, the Clostridium butyricum is administered with a potency of about 6.5×10⁹ CFU/dose to about 10×10⁹ CFU/dose. In embodiments, the Clostridium butyricum is administered with a potency of about 7.0×10⁹ CFU/dose to about 10×10⁹ CFU/dose. In embodiments, the Clostridium butyricum is administered with a potency of about 7.5×10⁹ CFU/dose to about 10×10⁹ CFU/dose. In embodiments, the Clostridium butyricum is administered with a potency of about 8.0×10⁹ CFU/dose to about 10×10⁹ CFU/dose. In embodiments, the Clostridium butyricum is administered with a potency of about 8.5×10⁹ CFU/dose to about 10×10⁹ CFU/dose. In embodiments, the Clostridium butyricum is administered with a potency of about 9.0×10⁹ CFU/dose to about 10×10⁹ CFU/dose. In embodiments, the Clostridium butyricum is administered with a potency of about 9.5×10⁹ CFU/dose to about 10×10⁹ CFU/dose.

In embodiments, the Clostridium butyricum is administered with a potency of about 0.1×10⁹ CFU/dose to about 9.5×10⁹ CFU/dose. In embodiments, the Clostridium butyricum is administered with a potency of about 0.1×10⁹ CFU/dose to about 9.0×10⁹ CFU/dose. In embodiments, the Clostridium butyricum is administered with a potency of about 0.1×10⁹ CFU/dose to about 8.5×10⁹ CFU/dose. In embodiments, the Clostridium butyricum is administered with a potency of about 0.1×10⁹ CFU/dose to about 8.0×10⁹ CFU/dose. In embodiments, the Clostridium butyricum is administered with a potency of about 0.1×10⁹ CFU/dose to about 7.5×10⁹ CFU/dose. In embodiments, the Clostridium butyricum is administered with a potency of about 0.1×10⁹ CFU/dose to about 7.0×10⁹ CFU/dose. In embodiments, the Clostridium butyricum is administered with a potency of about 0.1×10⁹ CFU/dose to about 6.5×10⁹ CFU/dose. In embodiments, the Clostridium butyricum is administered with a potency of about 0.1×10⁹ CFU/dose to about 6.0×10⁹ CFU/dose. In embodiments, the Clostridium butyricum is administered with a potency of about 0.1×10⁹ CFU/dose to about 5.5×10⁹ CFU/dose. In embodiments, the Clostridium butyricum is administered with a potency of about 0.1×10⁹ CFU/dose to about 5.0×10⁹ CFU/dose. In embodiments, the Clostridium butyricum is administered with a potency of about 0.1×10⁹ CFU/dose to about 4.5×10⁹ CFU/dose. In embodiments, the Clostridium butyricum is administered with a potency of about 0.1×10⁹ CFU/dose to about 4.0×10⁹ CFU/dose. In embodiments, the Clostridium butyricum is administered with a potency of about 0.1×10⁹ CFU/dose to about 3.5×10⁹ CFU/dose. In embodiments, the Clostridium butyricum is administered with a potency of about 0.1×10⁹ CFU/dose to about 3.0×10⁹ CFU/dose. In embodiments, the Clostridium butyricum is administered with a potency of about 0.1×10⁹ CFU/dose to about 2.5×10⁹ CFU/dose. In embodiments, the Clostridium butyricum is administered with a potency of about 0.1×10⁹ CFU/dose to about 2.0×10⁹ CFU/dose. In embodiments, the Clostridium butyricum is administered with a potency of about 0.1×10⁹ CFU/dose to about 1.5×10⁹ CFU/dose. In embodiments, the Clostridium butyricum is administered with a potency of about 0.1×10⁹ CFU/dose to about 1.0×10⁹ CFU/dose. In embodiments, the Clostridium butyricum is administered with a potency of about 0.1×10⁹ CFU/dose to about 0.5×10⁹ CFU/dose.

In embodiments, the Clostridium butyricum is administered with a potency of about 0.1×10⁹ CFU/dose, 0.5×10⁹ CFU/dose, 1.5×10⁹ CFU/dose, 2.0×10⁹ CFU/dose, about 2.5×10⁹ CFU/dose, 3.0×10⁹ CFU/dose, 3.5×10⁹ CFU/dose, 4.0×10⁹ CFU/dose, 4.5×10⁹ CFU/dose, 5.0×10⁹ CFU/dose, 5.5×10⁹ CFU/dose, about 6.0×10⁹ CFU/dose, 6.5×10⁹ CFU/dose, 7.0×10⁹ CFU/dose, 7.5×10⁹ CFU/dose, 8.0×10⁹ CFU/dose, 8.5×10⁹ CFU/dose, 9.0×10⁹ CFU/dose, 9.5×10⁹ CFU/dose, or 10×10⁹ CFU/dose. In embodiments, the Clostridium butyricum is administered with a potency of about 2.5×10⁹ CFU/dose. In embodiments, the Clostridium butyricum is administered with a potency of 2.5×10⁹ CFU/dose. In embodiments, the Clostridium butyricum is administered with a potency of about 5.0×10⁹ CFU/dose. In embodiments, the Clostridium butyricum is administered with a potency of 5.0×10⁹ CFU/dose. The potency of Clostridium butyricum may be any value or subrange within the recited ranges, including endpoints, or any range between any of the recited values.

For the dosages of Clostridium butyricum provided herein, in embodiments the dosage may be divided for administration. In embodiments, the dosage may be divided by about ½, about ⅓, about ¼, about ⅕, or about ⅙ for administration. In embodiments, the dosage may be divided by about ½. In embodiments, the dosage may be divided by about ⅓. In embodiments, the dosage may be divided by about ¼. In embodiments, the dosage may be divided by about ⅕. In embodiments, the dosage may be divided by about ⅙.

For the dosages of Clostridium butyricum provided herein, in embodiments, Clostridium butyricum may be administered for about 1 week to about 100 weeks. In embodiments, Clostridium butyricum may be administered for about 10 weeks to about 100 weeks. In embodiments, Clostridium butyricum may be administered for about 20 weeks to about 100 weeks. In embodiments, Clostridium butyricum may be administered for about 30 weeks to about 100 weeks. In embodiments, Clostridium butyricum may be administered for about 40 weeks to about 100 weeks. In embodiments, Clostridium butyricum may be administered for about 50 weeks to about 100 weeks. In embodiments, Clostridium butyricum may be administered for about 60 weeks to about 100 weeks. In embodiments, Clostridium butyricum may be administered for about 70 weeks to about 100 weeks. In embodiments, Clostridium butyricum may be administered for about 80 weeks to about 100 weeks. In embodiments, Clostridium butyricum may be administered for about 90 weeks to about 100 weeks.

In embodiments, Clostridium butyricum may be administered for about 1 week to about 90 weeks. In embodiments, Clostridium butyricum may be administered for about 1 week to about 80 weeks. In embodiments, Clostridium butyricum may be administered for about 1 week to about 70 weeks. In embodiments, Clostridium butyricum may be administered for about 1 week to about 60 weeks. In embodiments, Clostridium butyricum may be administered for about 1 week to about 50 weeks. In embodiments, Clostridium butyricum may be administered for about 1 week to about 40 weeks. In embodiments, Clostridium butyricum may be administered for about 1 week to about 30 weeks. In embodiments, Clostridium butyricum may be administered for about 1 week to about 20 weeks. In embodiments, Clostridium butyricum may be administered for about 1 week to about 10 weeks.

In embodiments, Clostridium butyricum may be administered for about 1 week, 10 weeks, 20 weeks, 30 weeks, 40 weeks, 50 weeks, 60 weeks, 70 weeks, 80 weeks, 90 weeks, or 100 weeks. The Clostridium butyricum administration time range may be any value or subrange within the recited ranges, including endpoints, or any range between any of the recited values.

For the dosages of Clostridium butyricum provided herein, in embodiments, Clostridium butyricum may be administered once daily for about 1 week to about 100 weeks. In embodiments, Clostridium butyricum may be administered once daily for about 10 weeks to about 100 weeks. In embodiments, Clostridium butyricum may be administered once daily for about 20 weeks to about 100 weeks. In embodiments, Clostridium butyricum may be administered once daily for about 30 weeks to about 100 weeks. In embodiments, Clostridium butyricum may be administered once daily for about 40 weeks to about 100 weeks. In embodiments, Clostridium butyricum may be administered once daily for about 50 weeks to about 100 weeks. In embodiments, Clostridium butyricum may be administered once daily for about 60 weeks to about 100 weeks. In embodiments, Clostridium butyricum may be administered once daily for about 70 weeks to about 100 weeks. In embodiments, Clostridium butyricum may be administered once daily for about 80 weeks to about 100 weeks.

In embodiments, Clostridium butyricum may be administered once daily for about 1 week to about 90 weeks. In embodiments, Clostridium butyricum may be administered once daily for about 1 week to about 80 weeks. In embodiments, Clostridium butyricum may be administered once daily for about 1 week to about 70 weeks. In embodiments, Clostridium butyricum may be administered once daily for about 1 week to about 60 weeks. In embodiments, Clostridium butyricum may be administered once daily for about 1 week to about 50 weeks. In embodiments, Clostridium butyricum may be administered once daily for about 1 week to about 40 weeks. In embodiments, Clostridium butyricum may be administered once daily for about 1 week to about 30 weeks. In embodiments, Clostridium butyricum may be administered once daily for about 1 week to about 20 weeks.

In embodiments, Clostridium butyricum may be administered once daily for about 1 week, about 10 weeks, about 20 weeks, about 30 weeks, about 40 weeks, about 50 weeks, about 60 weeks, about 70 weeks, about 80 weeks, about 90 weeks, or about 100 weeks. The Clostridium butyricum administration time points may be any value or subrange within the recited ranges, including endpoints, or any range between any of the recited values.

For the dosages of Clostridium butyricum provided herein, in embodiments, Clostridium butyricum may be administered twice daily for about 1 week to about 100 weeks. In embodiments, Clostridium butyricum may be administered twice daily for about 10 weeks to about 100 weeks. In embodiments, Clostridium butyricum may be administered twice daily for about 20 weeks to about 100 weeks. In embodiments, Clostridium butyricum may be administered twice daily for about 30 weeks to about 100 weeks. In embodiments, Clostridium butyricum may be administered twice daily for about 40 weeks to about 100 weeks. In embodiments, Clostridium butyricum may be administered twice daily for about 50 weeks to about 100 weeks. In embodiments, Clostridium butyricum may be administered twice daily for about 60 weeks to about 100 weeks. In embodiments, Clostridium butyricum may be administered twice daily for about 70 weeks to about 100 weeks. In embodiments, Clostridium butyricum may be administered twice daily for about 80 weeks to about 100 weeks.

In embodiments, Clostridium butyricum may be administered twice daily for about 1 week to about 90 weeks. In embodiments, Clostridium butyricum may be administered twice daily for about 1 week to about 80 weeks. In embodiments, Clostridium butyricum may be administered twice daily for about 1 week to about 70 weeks. In embodiments, Clostridium butyricum may be administered twice daily for about 1 week to about 60 weeks. In embodiments, Clostridium butyricum may be administered twice daily for about 1 week to about 50 weeks. In embodiments, Clostridium butyricum may be administered twice daily for about 1 week to about 40 weeks. In embodiments, Clostridium butyricum may be administered twice daily for about 1 week to about 30 weeks. In embodiments, Clostridium butyricum may be administered twice daily for about 1 week to about 20 weeks.

In embodiments, Clostridium butyricum may be administered twice daily for about 1 week, about 10 weeks, about 20 weeks, about 30 weeks, about 40 weeks, about 50 weeks, about 60 weeks, about 70 weeks, about 80 weeks, about 90 weeks, or about 100 weeks. The Clostridium butyricum administration time points may be any value or subrange within the recited ranges, including endpoints, or any range between any of the recited values.

For the dosages of Clostridium butyricum provided herein, in embodiments, Clostridium butyricum may be administered three times daily for about 1 week to about 100 weeks. In embodiments, Clostridium butyricum may be administered three times daily for about 10 weeks to about 100 weeks. In embodiments, Clostridium butyricum may be administered three times daily for about 20 weeks to about 100 weeks. In embodiments, Clostridium butyricum may be administered three times daily for about 30 weeks to about 100 weeks. In embodiments, Clostridium butyricum may be administered three times daily for about 40 weeks to about 100 weeks. In embodiments, Clostridium butyricum may be administered three times daily for about 50 weeks to about 100 weeks. In embodiments, Clostridium butyricum may be administered three times daily for about 60 weeks to about 100 weeks. In embodiments, Clostridium butyricum may be administered three times daily for about 70 weeks to about 100 weeks. In embodiments, Clostridium butyricum may be administered three times daily for about 80 weeks to about 100 weeks.

In embodiments, Clostridium butyricum may be administered three times daily for about 1 week to about 90 weeks. In embodiments, Clostridium butyricum may be administered three times daily for about 1 week to about 80 weeks. In embodiments, Clostridium butyricum may be administered three times daily for about 1 week to about 70 weeks. In embodiments, Clostridium butyricum may be administered three times daily for about 1 week to about 60 weeks. In embodiments, Clostridium butyricum may be administered three times daily for about 1 week to about 50 weeks. In embodiments, Clostridium butyricum may be administered three times daily for about 1 week to about 40 weeks. In embodiments, Clostridium butyricum may be administered three times daily for about 1 week to about 30 weeks. In embodiments, Clostridium butyricum may be administered three times daily for about 1 week to about 20 weeks.

In embodiments, Clostridium butyricum may be administered three times daily for about 1 week, about 10 weeks, about 20 weeks, about 30 weeks, about 40 weeks, about 50 weeks, about 60 weeks, about 70 weeks, about 80 weeks, about 90 weeks, or about 100 weeks. The Clostridium butyricum administration time points may be any value or subrange within the recited ranges, including endpoints, or any range between any of the recited values.

In embodiments, the Clostridium butyricum is administered once daily with a potency of about 0.1×10⁹ CFU/dose to about 10×10⁹ CFU/dose. In embodiments, the Clostridium butyricum is administered once daily with a potency of about 0.5×10⁹ CFU/dose to about 10×10⁹ CFU/dose. In embodiments, the Clostridium butyricum is administered once daily with a potency of about 1.0×10⁹ CFU/dose to about 10×10⁹ CFU/dose. In embodiments, the Clostridium butyricum is administered once daily with a potency of about 1.5×10⁹ CFU/dose to about 10×10⁹ CFU/dose. In embodiments, the Clostridium butyricum is administered once daily with a potency of about 2.0×10⁹ CFU/dose to about 10×10⁹ CFU/dose. In embodiments, the Clostridium butyricum is administered once daily with a potency of about 2.5×10⁹ CFU/dose to about 10×10⁹ CFU/dose. In embodiments, the Clostridium butyricum is administered once daily with a potency of about 3.0×10⁹ CFU/dose to about 10×10⁹ CFU/dose. In embodiments, the Clostridium butyricum is administered once daily with a potency of about 3.5×10⁹ CFU/dose to about 10×10⁹ CFU/dose. In embodiments, the Clostridium butyricum is administered once daily with a potency of about 4.0×10⁹ CFU/dose to about 10×10⁹ CFU/dose. In embodiments, the Clostridium butyricum is administered once daily with a potency of about 4.5×10⁹ CFU/dose to about 10×10⁹ CFU/dose. In embodiments, the Clostridium butyricum is administered once daily with a potency of about 5.0×10⁹ CFU/dose to about 10×10⁹ CFU/dose. In embodiments, the Clostridium butyricum is administered once daily with a potency of about 5.5×10⁹ CFU/dose to about 10×10⁹ CFU/dose. In embodiments, the Clostridium butyricum is administered once daily with a potency of about 6.0×10⁹ CFU/dose to about 10×10⁹ CFU/dose. In embodiments, the Clostridium butyricum is administered once daily with a potency of about 6.5×10⁹ CFU/dose to about 10×10⁹ CFU/dose. In embodiments, the Clostridium butyricum is administered once daily with a potency of about 7.0×10⁹ CFU/dose to about 10×10⁹ CFU/dose. In embodiments, the Clostridium butyricum is administered once daily with a potency of about 7.5×10⁹ CFU/dose to about 10×10⁹ CFU/dose. In embodiments, the Clostridium butyricum is administered once daily with a potency of about 8.0×10⁹ CFU/dose to about 10×10⁹ CFU/dose. In embodiments, the Clostridium butyricum is administered once daily with a potency of about 8.5×10⁹ CFU/dose to about 10×10⁹ CFU/dose. In embodiments, the Clostridium butyricum is administered once daily with a potency of about 9.0×10⁹ CFU/dose to about 10×10⁹ CFU/dose.

In embodiments, the Clostridium butyricum is administered once daily with a potency of about 0.1×10⁹ CFU/dose to about 9.5×10⁹ CFU/dose. In embodiments, the Clostridium butyricum is administered once daily with a potency of about 0.1×10⁹ CFU/dose to about 9.0×10⁹ CFU/dose. In embodiments, the Clostridium butyricum is administered once daily with a potency of about 0.1×10⁹ CFU/dose to about 8.5×10⁹ CFU/dose. In embodiments, the Clostridium butyricum is administered once daily with a potency of about 0.1×10⁹ CFU/dose to about 8.0×10⁹ CFU/dose. In embodiments, the Clostridium butyricum is administered once daily with a potency of about 0.1×10⁹ CFU/dose to about 7.5×10⁹ CFU/dose. In embodiments, the Clostridium butyricum is administered once daily with a potency of about 0.1×10⁹ CFU/dose to about 7.0×10⁹ CFU/dose. In embodiments, the Clostridium butyricum is administered once daily with a potency of about 0.1×10⁹ CFU/dose to about 6.5×10⁹ CFU/dose. In embodiments, the Clostridium butyricum is administered once daily with a potency of about 0.1×10⁹ CFU/dose to about 6.0×10⁹ CFU/dose. In embodiments, the Clostridium butyricum is administered once daily with a potency of about 0.1×10⁹ CFU/dose to about 5.5×10⁹ CFU/dose. In embodiments, the Clostridium butyricum is administered once daily with a potency of about 0.1×10⁹ CFU/dose to about 5.0×10⁹ CFU/dose. In embodiments, the Clostridium butyricum is administered once daily with a potency of about 0.1×10⁹ CFU/dose to about 4.5×10⁹ CFU/dose. In embodiments, the Clostridium butyricum is administered once daily with a potency of about 0.1×10⁹ CFU/dose to about 4.0×10⁹ CFU/dose. In embodiments, the Clostridium butyricum is administered once daily with a potency of about 0.1×10⁹ CFU/dose to about 3.5×10⁹ CFU/dose. In embodiments, the Clostridium butyricum is administered once daily with a potency of about 0.1×10⁹ CFU/dose to about 3.0×10⁹ CFU/dose. In embodiments, the Clostridium butyricum is administered once daily with a potency of about 0.1×10⁹ CFU/dose to about 2.5×10⁹ CFU/dose. In embodiments, the Clostridium butyricum is administered once daily with a potency of about 0.1×10⁹ CFU/dose to about 2.0×10⁹ CFU/dose. In embodiments, the Clostridium butyricum is administered once daily with a potency of about 0.1×10⁹ CFU/dose to about 1.5×10⁹ CFU/dose. In embodiments, the Clostridium butyricum is administered once daily with a potency of about 0.1×10⁹ CFU/dose to about 1.0×10⁹ CFU/dose.

In embodiments, the Clostridium butyricum is administered once daily with a potency of about 0.1×10⁹ CFU/dose, 0.5×10⁹ CFU/dose, 1.5×10⁹ CFU/dose, 2.0×10⁹ CFU/dose, 2.5×10⁹ CFU/dose, 3.0×10⁹ CFU/dose, 3.5×10⁹ CFU/dose, 4.0×10⁹ CFU/dose, 4.5×10⁹ CFU/dose, 5.0×10⁹ CFU/dose, 5.5×10⁹ CFU/dose, 6.0×10⁹ CFU/dose, 6.5×10⁹ CFU/dose, 7.0×10⁹ CFU/dose, 7.5×10⁹ CFU/dose, 8.0×10⁹ CFU/dose, 8.5×10⁹ CFU/dose, 9.0×10⁹ CFU/dose, 9.5×10⁹ CFU/dose or 10×10⁹ CFU/dose. In embodiments, the Clostridium butyricum is administered once daily with a potency of about 2.5×10⁹ CFU/dose. In embodiments, the Clostridium butyricum is administered once daily with a potency of 2.5×10⁹ CFU/dose. In embodiments, the Clostridium butyricum is administered once daily with a potency of about 5.0×10⁹ CFU/dose. In embodiments, the Clostridium butyricum is administered once daily with a potency of 5.0×10⁹ CFU/dose. The potency of CBM588 LBP may be any value or subrange within the recited ranges, including endpoints, or any range between any of the recited values.

In embodiments, the Clostridium butyricum is administered twice daily with a potency of about 0.1×10⁹ CFU/dose to about 10×10⁹ CFU/dose. In embodiments, the Clostridium butyricum is administered twice daily with a potency of about 0.5×10⁹ CFU/dose to about 10×10⁹ CFU/dose. In embodiments, the Clostridium butyricum is administered twice daily with a potency of about 1.0×10⁹ CFU/dose to about 10×10⁹ CFU/dose. In embodiments, the Clostridium butyricum is administered twice daily with a potency of about 1.5×10⁹ CFU/dose to about 10×10⁹ CFU/dose. In embodiments, the Clostridium butyricum is administered twice daily with a potency of about 2.0×10⁹ CFU/dose to about 10×10⁹ CFU/dose. In embodiments, the Clostridium butyricum is administered twice daily with a potency of about 2.5×10⁹ CFU/dose to about 10×10⁹ CFU/dose. In embodiments, the Clostridium butyricum is administered twice daily with a potency of about 3.0×10⁹ CFU/dose to about 10×10⁹ CFU/dose. In embodiments, the Clostridium butyricum is administered twice daily with a potency of about 3.5×10⁹ CFU/dose to about 10×10⁹ CFU/dose. In embodiments, the Clostridium butyricum is administered twice daily with a potency of about 4.0×10⁹ CFU/dose to about 10×10⁹ CFU/dose. In embodiments, the Clostridium butyricum is administered twice daily with a potency of about 4.5×10⁹ CFU/dose to about 10×10⁹ CFU/dose. In embodiments, the Clostridium butyricum is administered twice daily with a potency of about 5.0×10⁹ CFU/dose to about 10×10⁹ CFU/dose. In embodiments, the Clostridium butyricum is administered twice daily with a potency of about 5.5×10⁹ CFU/dose to about 10×10⁹ CFU/dose. In embodiments, the Clostridium butyricum is administered twice daily with a potency of about 6.0×10⁹ CFU/dose to about 10×10⁹ CFU/dose. In embodiments, the Clostridium butyricum is administered twice daily with a potency of about 6.5×10⁹ CFU/dose to about 10×10⁹ CFU/dose. In embodiments, the Clostridium butyricum is administered twice daily with a potency of about 7.0×10⁹ CFU/dose to about 10×10⁹ CFU/dose. In embodiments, the Clostridium butyricum is administered twice daily with a potency of about 7.5×10⁹ CFU/dose to about 10×10⁹ CFU/dose. In embodiments, the Clostridium butyricum is administered twice daily with a potency of about 8.0×10⁹ CFU/dose to about 10×10⁹ CFU/dose. In embodiments, the Clostridium butyricum is administered twice daily with a potency of about 8.5×10⁹ CFU/dose to about 10×10⁹ CFU/dose. In embodiments, the Clostridium butyricum is administered twice daily with a potency of about 9.0×10⁹ CFU/dose to about 10×10⁹ CFU/dose.

In embodiments, the Clostridium butyricum is administered twice daily with a potency of about 0.1×10⁹ CFU/dose to about 9.5×10⁹ CFU/dose. In embodiments, the Clostridium butyricum is administered twice daily with a potency of about 0.1×10⁹ CFU/dose to about 9.0×10⁹ CFU/dose. In embodiments, the Clostridium butyricum is administered twice daily with a potency of about 0.1×10⁹ CFU/dose to about 8.5×10⁹ CFU/dose. In embodiments, the Clostridium butyricum is administered twice daily with a potency of about 0.1×10⁹ CFU/dose to about 8.0×10⁹ CFU/dose. In embodiments, the Clostridium butyricum is administered twice daily with a potency of about 0.1×10⁹ CFU/dose to about 7.5×10⁹ CFU/dose. In embodiments, the Clostridium butyricum is administered twice daily with a potency of about 0.1×10⁹ CFU/dose to about 7.0×10⁹ CFU/dose. In embodiments, the Clostridium butyricum is administered twice daily with a potency of about 0.1×10⁹ CFU/dose to about 6.5×10⁹ CFU/dose. In embodiments, the Clostridium butyricum is administered twice daily with a potency of about 0.1×10⁹ CFU/dose to about 6.0×10⁹ CFU/dose. In embodiments, the Clostridium butyricum is administered twice daily with a potency of about 0.1×10⁹ CFU/dose to about 5.5×10⁹ CFU/dose. In embodiments, the Clostridium butyricum is administered twice daily with a potency of about 0.1×10⁹ CFU/dose to about 5.0×10⁹ CFU/dose. In embodiments, the Clostridium butyricum is administered twice daily with a potency of about 0.1×10⁹ CFU/dose to about 4.5×10⁹ CFU/dose. In embodiments, the Clostridium butyricum is administered twice daily with a potency of about 0.1×10⁹ CFU/dose to about 4.0×10⁹ CFU/dose. In embodiments, the Clostridium butyricum is administered twice daily with a potency of about 0.1×10⁹ CFU/dose to about 3.5×10⁹ CFU/dose. In embodiments, the Clostridium butyricum is administered twice daily with a potency of about 0.1×10⁹ CFU/dose to about 3.0×10⁹ CFU/dose. In embodiments, the Clostridium butyricum is administered twice daily with a potency of about 0.1×10⁹ CFU/dose to about 2.5×10⁹ CFU/dose. In embodiments, the Clostridium butyricum is administered twice daily with a potency of about 0.1×10⁹ CFU/dose to about 2.0×10⁹ CFU/dose. In embodiments, the Clostridium butyricum is administered twice daily with a potency of about 0.1×10⁹ CFU/dose to about 1.5×10⁹ CFU/dose. In embodiments, the Clostridium butyricum is administered twice daily with a potency of about 0.1×10⁹ CFU/dose to about 1.0×10⁹ CFU/dose.

In embodiments, the Clostridium butyricum is administered twice daily with a potency of about 0.1×10⁹ CFU/dose, 0.5×10⁹ CFU/dose, 1.5×10⁹ CFU/dose, 2.0×10⁹ CFU/dose, 2.5×10⁹ CFU/dose, 3.0×10⁹ CFU/dose, 3.5×10⁹ CFU/dose, 4.0×10⁹ CFU/dose, 4.5×10⁹ CFU/dose, 5.0×10⁹ CFU/dose, 5.5×10⁹ CFU/dose, 6.0×10⁹ CFU/dose, 6.5×10⁹ CFU/dose, 7.0×10⁹ CFU/dose, 7.5×10⁹ CFU/dose, 8.0×10⁹ CFU/dose, 8.5×10⁹ CFU/dose, 9.0×10⁹ CFU/dose, 9.5×10⁹ CFU/dose, or about 10×10⁹ CFU/dose. In embodiments, the Clostridium butyricum is administered twice daily with a potency of about 2.5×10⁹ CFU/dose. In embodiments, the Clostridium butyricum is administered twice daily with a potency of 2.5×10⁹ CFU/dose. In embodiments, the Clostridium butyricum is administered twice daily with a potency of about 5.0×10⁹ CFU/dose. In embodiments, the Clostridium butyricum is administered twice daily with a potency of 5.0×10⁹ CFU/dose. The potency of Clostridium butyricum may be any value or subrange within the recited ranges, including endpoints, or any range between any of the recited values.

In embodiments, the Clostridium butyricum is administered three times daily with a potency of about 0.1×10⁹ CFU/dose to about 10×10⁹ CFU/dose. In embodiments, the Clostridium butyricum is administered three times daily with a potency of about 0.5×10⁹ CFU/dose to about 10×10⁹ CFU/dose. In embodiments, the Clostridium butyricum is administered three times daily with a potency of about 1.0×10⁹ CFU/dose to about 10×10⁹ CFU/dose. In embodiments, the Clostridium butyricum is administered three times daily with a potency of about 1.5×10⁹ CFU/dose to about 10×10⁹ CFU/dose. In embodiments, the Clostridium butyricum is administered three times daily with a potency of about 2.0×10⁹ CFU/dose to about 10×10⁹ CFU/dose. In embodiments, the Clostridium butyricum is administered three times daily with a potency of about 2.5×10⁹ CFU/dose to about 10×10⁹ CFU/dose. In embodiments, the Clostridium butyricum is administered three times daily with a potency of about 3.0×10⁹ CFU/dose to about 10×10⁹ CFU/dose. In embodiments, the Clostridium butyricum is administered three times daily with a potency of about 3.5×10⁹ CFU/dose to about 10×10⁹ CFU/dose. In embodiments, the Clostridium butyricum is administered three times daily with a potency of about 4.0×10⁹ CFU/dose to about 10×10⁹ CFU/dose. In embodiments, the Clostridium butyricum is administered three times daily with a potency of about 4.5×10⁹ CFU/dose to about 10×10⁹ CFU/dose. In embodiments, the Clostridium butyricum is administered three times daily with a potency of about 5.0×10⁹ CFU/dose to about 10×10⁹ CFU/dose. In embodiments, the Clostridium butyricum is administered three times daily with a potency of about 5.5×10⁹ CFU/dose to about 10×10⁹ CFU/dose. In embodiments, the Clostridium butyricum is administered three times daily with a potency of about 6.0×10⁹ CFU/dose to about 10×10⁹ CFU/dose. In embodiments, the Clostridium butyricum is administered three times daily with a potency of about 6.5×10⁹ CFU/dose to about 10×10⁹ CFU/dose. In embodiments, the Clostridium butyricum is administered three times daily with a potency of about 7.0×10⁹ CFU/dose to about 10×10⁹ CFU/dose. In embodiments, the Clostridium butyricum is administered three times daily with a potency of about 7.5×10⁹ CFU/dose to about 10×10⁹ CFU/dose. In embodiments, the Clostridium butyricum is administered three times daily with a potency of about 8.0×10⁹ CFU/dose to about 10×10⁹ CFU/dose. In embodiments, the Clostridium butyricum is administered three times daily with a potency of about 8.5×10⁹ CFU/dose to about 10×10⁹ CFU/dose. In embodiments, the Clostridium butyricum is administered three times daily with a potency of about 9.0×10⁹ CFU/dose to about 10×10⁹ CFU/dose.

In embodiments, the Clostridium butyricum is administered three times daily with a potency of about 0.1×10⁹ CFU/dose to about 9.5×10⁹ CFU/dose. In embodiments, the Clostridium butyricum is administered three times daily with a potency of about 0.1×10⁹ CFU/dose to about 9.0×10⁹ CFU/dose. In embodiments, the Clostridium butyricum is administered three times daily with a potency of about 0.1×10⁹ CFU/dose to about 8.5×10⁹ CFU/dose. In embodiments, the Clostridium butyricum is administered three times daily with a potency of about 0.1×10⁹ CFU/dose to about 8.0×10⁹ CFU/dose. In embodiments, the Clostridium butyricum is administered three times daily with a potency of about 0.1×10⁹ CFU/dose to about 7.5×10⁹ CFU/dose. In embodiments, the Clostridium butyricum is administered three times daily with a potency of about 0.1×10⁹ CFU/dose to about 7.0×10⁹ CFU/dose. In embodiments, the Clostridium butyricum is administered three times daily with a potency of about 0.1×10⁹ CFU/dose to about 6.5×10⁹ CFU/dose. In embodiments, the Clostridium butyricum is administered three times daily with a potency of about 0.1×10⁹ CFU/dose to about 6.0×10⁹ CFU/dose. In embodiments, the Clostridium butyricum is administered three times daily with a potency of about 0.1×10⁹ CFU/dose to about 5.5×10⁹ CFU/dose. In embodiments, the Clostridium butyricum is administered three times daily with a potency of about 0.1×10⁹ CFU/dose to about 5.0×10⁹ CFU/dose. In embodiments, the Clostridium butyricum is administered three times daily with a potency of about 0.1×10⁹ CFU/dose to about 4.5×10⁹ CFU/dose. In embodiments, the Clostridium butyricum is administered three times daily with a potency of about 0.1×10⁹ CFU/dose to about 4.0×10⁹ CFU/dose. In embodiments, the Clostridium butyricum is administered three times daily with a potency of about 0.1×10⁹ CFU/dose to about 3.5×10⁹ CFU/dose. In embodiments, the Clostridium butyricum is administered three times daily with a potency of about 0.1×10⁹ CFU/dose to about 3.0×10⁹ CFU/dose. In embodiments, the Clostridium butyricum is administered three times daily with a potency of about 0.1×10⁹ CFU/dose to about 2.5×10⁹ CFU/dose. In embodiments, the Clostridium butyricum is administered three times daily with a potency of about 0.1×10⁹ CFU/dose to about 2.0×10⁹ CFU/dose. In embodiments, the Clostridium butyricum is administered three times daily with a potency of about 0.1×10⁹ CFU/dose to about 1.5×10⁹ CFU/dose. In embodiments, the Clostridium butyricum is administered three times daily with a potency of about 0.1×10⁹ CFU/dose to about 1.0×10⁹ CFU/dose.

In embodiments, the Clostridium butyricum is administered three times daily with a potency of about 0.1×10⁹ CFU/dose, 0.5×10⁹ CFU/dose, 1.5×10⁹ CFU/dose, 2.0×10⁹ CFU/dose, 2.5×10⁹ CFU/dose, 3.0×10⁹ CFU/dose, 3.5×10⁹ CFU/dose, 4.0×10⁹ CFU/dose, 4.5×10⁹ CFU/dose, 5.0×10⁹ CFU/dose, 5.5×10⁹ CFU/dose, 6.0×10⁹ CFU/dose, 6.5×10⁹ CFU/dose, 7.0×10⁹ CFU/dose, 7.5×10⁹ CFU/dose, 8.0×10⁹ CFU/dose, 8.5×10⁹ CFU/dose, 9.0×10⁹ CFU/dose, 9.5×10⁹ CFU/dose or about 10×10⁹ CFU/dose. In embodiments, the Clostridium butyricum is administered three times daily with a potency of about 2.5×10⁹ CFU/dose. In embodiments, the Clostridium butyricum is administered three times daily with a potency of 2.5×10⁹ CFU/dose. In embodiments, the Clostridium butyricum is administered three times daily with a potency of about 5.0×10⁹ CFU/dose. In embodiments, the Clostridium butyricum is administered three times daily with a potency of 5.0×10⁹ CFU/dose. The potency of CBM588 LBP may be any value or subrange within the recited ranges, including endpoints, or any range between any of the recited values.

In embodiments, the Clostridium butyricum may be administered at about 5×10⁹ CFU/dose. In embodiments, the Clostridium butyricum may be administered at 5×10⁹ CFU/dose. In embodiments, the 5×10⁹ CFU/dose of CBM588 LBP may be administered twice daily.

### Numbered Embodiments

Embodiment 1. A method of treating or preventing a graft-versus-host disease (GVHD) in a subject, said method comprising administering to said subject a therapeutically effective amount of Clostridium butyricum.

Embodiment 2. The method of embodiment 1, wherein said Clostridium butyricum is a live biotherapeutic product.

Embodiment 3. The method of embodiment 1 or 2, wherein said Clostridium butyricum live biotherapeutic product is Clostridium butyricum MIYAIRI 588 live biotherapeutic product (CBM588 LBP).

Embodiment 4. The method of any one of embodiments 1 to 3, wherein said subject is a subject undergoing allogeneic hematopoietic stem cell transplantation.

Embodiment 5. The method of any one of embodiments 1 to 4, wherein said subject is a cancer subject undergoing allogeneic hematopoietic stem cell transplantation.

Embodiment 6. The method of any one of embodiments 1 to 5, wherein said cancer subject is a hematological cancer subject.

Embodiment 7. The method of any one of embodiments 1 to 6, wherein said hematological cancer subject is leukemia subject, lymphoma subject, myeloma subject or subject with myelodysplastic syndrome.

Embodiment 8. The method of any one of embodiments 1 to 4, wherein said subject suffers from non-cancer hematological disorder.

Embodiment 9. The method of embodiment 8, wherein said non-cancer hematological disorder is a sickle cell disorder.

Embodiment 10. The method of any one of embodiments 1 to 9, wherein the dose of CBM588 LBP is about 50 mg per day to about 250 mg per day.

Embodiment 11. The method of embodiment 10, wherein the dose of CBM588 LBP is about 100 mg per day to about 200 mg per day.

Embodiment 12. The method of embodiment 11, wherein the dose of CBM588 LBP is about 120 mg per day to about 180 mg per day.

Embodiment 13. The method of embodiment 12 wherein the dose of CBM588 LBP is about 160 mg per day.

Embodiment 14. The method of any one of embodiments 1 to 13, wherein CBM588 LBP is administered orally.

Embodiment 15. The method of any one of embodiments 1 to 13, wherein CBM588 LBP is administered once a day, twice a day or three times a day.

Embodiment 16. The method of any one of embodiments 1 to 15, wherein CBM588 LBP is administered for at least 28 days.

Embodiment 17. The method of any one of embodiments 1 to 15, wherein CBM588 LBP is administered for at least 14 days.

Embodiment 18. A method of increasing gut biodiversity in a subject, said method comprising administering to said subject a therapeutically effective amount of Clostridium butyricum.

Embodiment 19. The method of claim 18, wherein said Clostridium butyricum is a live biotherapeutic product.

Embodiment 20. The method of embodiment 18 or 19, wherein said Clostridium butyricum live biotherapeutic product is Clostridium butyricum MIYAIRI 588 live biotherapeutic product (CBM588 LBP).

Embodiment 21. The method of any one of embodiments 18 to 20, wherein said subject is a subject undergoing allogeneic hematopoietic stem cell transplantation.

Embodiment 22. The method of any one of embodiments 18 to 21, wherein said subject is a cancer subject undergoing allogeneic hematopoietic stem cell transplantation.

Embodiment 23. The method of any one of embodiments 18 to 22, wherein said cancer subject is a hematological cancer subject.

Embodiment 24. The method of any one of embodiments 18 to 23, wherein said hematological cancer subject is leukemia subject, lymphoma subject, myeloma subject or subject with myelodysplastic syndrome.

Embodiment 25. The method of any one of embodiments 18 to 20, wherein said subject suffers from non-cancer hematological disorder.

Embodiment 26. The method of embodiment 25, wherein said non-cancer hematological disorder is a sickle cell disorder.

Embodiment 27. A method of improving clinical outcome of a subject undergoing allogeneic hematopoietic stem cell transplantation, said method comprising administering to said subject a therapeutically effective amount of Clostridium butyricum.

Embodiment 28. The method of embodiment 27, wherein said Clostridium butyricum is a live biotherapeutic product.

Embodiment 29. The method of embodiment 27 or 28, wherein said Clostridium butyricum live biotherapeutic product is Clostridium butyricum MIYAIRI 588 live biotherapeutic product (CBM588 LBP).

Embodiment 30. The method of any one of embodiments 27 to 29, wherein said subject is a subject undergoing allogeneic hematopoietic stem cell transplantation.

Embodiment 31. The method of any one of embodiments 27 to 30, wherein said subject is a cancer subject undergoing allogeneic hematopoietic stem cell transplantation.

Embodiment 32. The method of any one of embodiments 27 to 31, wherein said cancer subject is a hematological cancer subject.

Embodiment 33. The method of any one of embodiments 27 to 32, wherein said hematological cancer subject is leukemia subject, lymphoma subject, myeloma subject or subject with myelodysplastic syndrome.

Embodiment 34. The method of any one of embodiments 27 to 30, wherein said subject suffers from non-cancer hematological disorder.

Embodiment 35. The method of embodiment 34, wherein said non-cancer hematological disorder is a sickle cell disorder.

Embodiment 36. The method of embodiment 27, wherein said clinical outcome is acute GVHD or an infection.

Embodiment 37. A pharmaceutical composition comprising Clostridium butyricum in a dosage form.

Embodiment 38. The pharmaceutical composition of embodiment 37, wherein said Clostridium butyricum is a live biotherapeutic product.

Embodiment 39. The pharmaceutical composition of embodiment 38, wherein said Clostridium butyricum live biotherapeutic product is Clostridium butyricum MIYAIRI 588 live biotherapeutic product (CBM588 LBP).

### Examples

### Example 1: Randomized Open Label Pilot Study of Clostridium butyricum MIYAIRI 588 (CBM588) in Recipients of Allgeneic Hematopoiei Cell Transplantation

### Background of Studies Described Herein

Allogeneic hematopoietic stem cell transplantation (HCT) is a curative treatment option for a wide range of hematological and non-hematological disorders. Despite recent advances in supportive care, graft-versus-host disease (GVHD), infection, and relapse of the underlying hematologic disease remain the major causes of morbidity and mortality in HCT recipients.

The gut microbiota has been shown to play an important role in maintaining intestinal homeostasis by regulating the maturation of the mucosal immune system, which constitutes an immune barrier for the integrity of the intestinal tract. Allogeneic HCT patients are a unique population in which the gut ecosystem is already perturbed by previous interventions including chemotherapy and antibacterial therapy, and then further impaired as a result of intensive conditioning regimens and donor T cell assault (GVHD). In recent years, the role of the human GI microbiota in GVHD has been considered in observational studies. However, there have been no prospective trials to date demonstrating a clinical benefit of dietary interventions specifically designed to alter the microbiota of HCT recipients.

CBM588 is a novel Live Biotherapeutic Product (LBP) that contains the bacterium Clostridium butyricum MIYAIRI 588 (CBM588). CBM588 produces short chain organic acids (SCFAs), most notably butyric acid, which plays a key role in the maintenance of colonic homeostasis by regulating fluid and electrolyte uptake, epithelial cell growth, and inflammatory responses. CBM588 has a spectrum of activities that also includes direct antagonism of enteric pathogens, stimulation of the growth of beneficial bacteria, including bifidobacteria and lactobacilli, and recovery of the normal anaerobic microflora following disruption of the intestinal ecosystem, typically by antibiotic use.

Applicants therefore proposed assessing Clostridium butyricum MIYAIRI 588 live biotherapeutic product's (CBM588 LBP) safety and to determine its ability to increase gut biodiversity in alloHCT patients and to improve clinical outcomes such as preventing or avoiding GVHD and infections in these patients.

### Methods

HCT procedures were performed according to the City of Hope HCT Standard of Procedures (SOP).

Stool samples and serum correlatives were collected weekly for the first 6 weeks of therapy (day −8 through day +28), and monthly×2 thereafter (days 60 and 100). Fecal samples were processed using protocols established by the Earth Microbiome Project (EMP). Briefly, DNA from 250 mg of fecal material for each sample was extracted using the DNeasy PowerSoil kit (MoBio Laboratories, Qiagen Company, Carlsbad, CA). The manufacturer's protocol was followed with the exception of a 10 minute incubation at 65 C after the addition of solution C1 per the EMP protocol. 16S amplicon libraries with barcoded adapters corresponding to the Illumina chemistry were prepared from the extracted DNA using previously described methods. Each library was quantified with qPCR (Kapa Biosystems; Wilmington, MA). The quantified libraries were pooled at equimolar concentrations. The pool was quantified and run on the Illumina MiSeq using version 3 chemistry (Illumina Inc.; San Diego, CA).

Sequence reads were processed by Mothur software, as described in MiSeq SOP, assembled in OUTs, taxonomically annotated to the level of genus and used to construct Bray-Curtis dissimilarity matrix. The similarity of samples was visualized by PCoA and further confirmed by ANOSIM tests, differentially abundant taxa were determined by METASTATS software.

Processed fecal DNA was subject to PCR using universal primers. The PCR amplicons were sequenced, rarefied to 10,000 sequences/sample and low-quality sequences were trimmed. Chimeric sequences were removed and assembled in 7,097 operating taxonomic units (OTUs) and taxonomically annotated to the genera level. OTU size ranged from 1 sequence for median and minimal sizes, 37 for average sizes, and 30,878 for maximum sizes. The OTUs were used to assess the structure, membership, and dynamics of the gut microbial community. OTU abundances were standardized and used to calculate distances between samples using Bray-Curtis dissimilarity measure, and visualized by PCoA plot. Distribution of samples confirmed that the structure of gut microbiota was patient specific (ANOSIM, p=0.001) and that the treatment response was among significant factors affecting sample separation (ANOSIM, p=0.01).

### CBM588 LBP

CBM588 is a strain of Clostridium butyricum used commercially in Japan as a live biotherapeutic product in humans and a feed additive in animals. CBM588 was authorized by the European Union as a novel food ingredient in 2014, and as a feed additive for turkeys, chickens, and related minor avian species. In a pediatric study including 110 children with upper respiratory tract infection or gastroenteritis, CBM588 administered as an LBP was safe and well-tolerated. Furthermore, the incidence of antibiotic-related diarrhea was markedly reduced in patients who received CBM588 LBP (59% vs 5%). In a study of ulcerative colitis, CBM588 LBP was administered at a dose of 60 mg oral tid.

### Eligibility Criteria

Inclusion Criteria: Patients age ≥18 years, scheduled to undergo HCT from an 8/8 or 7/8 HLA matched related/unrelated donor with reduced intencity conditioning (RIC) with adequate organ functions and performance status were eligible.

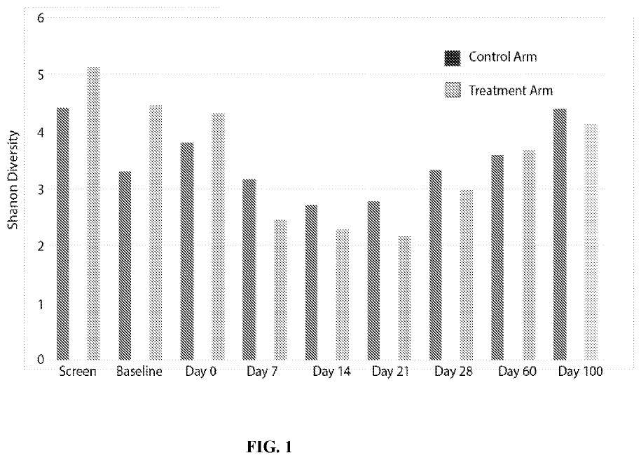

Following the patient safety lead-in (SLI; n=6), 30 patients were randomized (1:1 ratio) to receive either standard peri-transplant supportive care alone (control arm) or with CBM588 (treatment arm, open label) at the fixed dose of 160 mg orally (2×/day) from day −8 or hospital admission until day +28 of discharge (FIG. 1). Patients received prophylactic antibiotics per intuitional SOPs.

### Study Objectives

The primary objective was to determine the safety, feasibility, biological activities, and preliminart efficacy of CBM588 in HCT recipients. Secondary objectives were to evaluate/compare microbiome diversity, the incidence and severity of adverse events (AE), HCT outcomes including GVHD, infections, and immune-biologic endpoints such as cytokines, cellular immune reconstitution, and metabolomics such as butyrate and other short-chain fatty acids. Feasibility was defined as the ability to consume CBM588 for 14 days during the SLI phase. For microbiome analysis, DNA was isolated from weekly collected stool samples and the V4 region of the bacterial 16S rRNA gene from each total DNA sample was amplified.

### Clinical Trial Information

The study completed its accrual of 36 subjects (20 female) at the median age of 66 years (range 34-77) with at least 3 months of follow up from HCT. The indication for HCT was leukemia (n=22), MDS (n=5), lymphoma (n=3), myeloma (n=3), or other (n=3). All but one patient received fludarabine/melphalan-based RIC and tacrolimus/sirolimus-based GVHD prophylaxis. Graft source was peripheral blood stem cells from a matched related (n=13) or unrelated (n=23) donor (Table 1).

TABLE 1
Patient and HOT characteristics

| | Lead-in | CBM588 | Control | Total |
|---|---|---|---|---|
| | (N = 6) | (N = 15) | (N = 15) | (N = 36) |
| Age | | | | |
| Median | 70 (58-71) | 66 (34-77) | 64 (43-75) | 66 (34-77) |
| Gender | | | | |
| Female | 4 (66.7%) | 6 (40%) | 10 (66.7%) | 20 (55.6%) |
| Primary diagnosis | | | | |
| AML | 4 (66.7%) | 4 (26.7%) | 8 (53.3%) | 16 (44.4%) |
| ALL | 2 (33.3%) | 3 (20%) | 1 (6.7%) | 6 (16.7%) |
| MDS | 0 (0%) | 2 (13.3%) | 3 (20%) | 5 (13.9%) |
| NHL/MCL | 0 (0%) | 3 (20%) | 0 (0%) | 3 (8.3%) |
| MM | 0 (0%) | 2 (13.3%) | 1 (6.7%) | 3 (8.3%) |
| others | 0 (0%) | 1 (0%) | 2 (13.3%) | 3 (8.3%) |
| HCTCI | | | | |
| 0-1 | 5 (83.3%) | 13 (86.7%) | 14 (93.3%) | 32 (88.9%) |
| >=2 | 1 (16.7%) | 2 (13.4%) | 1 (6.7%) | 4 (11.2%) |
| KPS conditioning | | | | |
| 70-80 | 0 (0%) | 6 (40%) | 1 (6.7%) | 7 (19.5%) |
| 90-100 | 6 (100%) | 9 (60%) | 14 (93.3%) | 29 (80%) |
| Donor Type | | | | |
| Related | 1 (16.7%) | 7 (46.7%) | 5 (33.3%) | 13 (36.1%) |
| Unrelated | 5 (83.3%) | 8 (53.3%) | 10 (66.7%) | 23 (63.9%) |

One patient assigned to a treatment arm declined to receive CBM588 before the first dose, but remained on the study with clinical data/biospecimen collections and safety/feasibility/biologic endpoints being analyzed as treated for this patient. All the other patients assigned to the Treatment arm (n=21, including the patient on SLI segment) were able to take the prescribed study drug. CBM588 was well tolerated in HCT recipients with the median doses of 52 (range: 0-55), and 19 of 21 subjects (90.5%) consumed at least 14 days of the study drug. There were no serious adverse events (SAE) related to CBM588. The overall AEs and infection- or GI-specific AEs were similar between the Treatment and Control arms. All but one patient (who died of sepsis in the Control arm—on day 8) engrafted with a median of 15 days for neutrophils. The 100-day non-relapse mortality (NRM) was 0% in the Treatment and 6.7% in the Control arm. The one-year survival was 93% in the Treatment arm and 80% in the Control arm.

According to the intent-to-treat principle, acute GVHD (grade 2-4) in 4 of 15 patients in the Treatment arm and 5 of 15 in the Control arm. The lower GI GVHD was observed in 2 patients in the Treatment arm and 4 patients in the Control arm. As treated analyses showed, the overall grade 2-4 GVHD was observed in 3 of 14 patients (21.4%) with the use of CBM588 and 6 of 16 (37.5%) without CBM588. When the analysis was focused on lower GI GVHD, only one case of lower GI GVHD was seen in the CBM588 arm while 5 patients developed lower GI GVHD in the observation arm. (Table 2). The results showed no significant safety concerns or detection of serious adverse events (SAE) related to CBM588.

TABLE 2
GVHD outcomes

| | Lead-in | CBM588 | Control |
|---|---|---|---|
| | (N = 6) | (N = 15) | (N = 15) |
| aGVHD | | | |
| II | 1 (16.7%) | 3 (20%) | 5 (33.3%) |
| III | 1 (16.7%) | 1 (6.7%) | 0 (0%) |
| Lower GI GVHD | | | |
| Stage 1 | 0 (0%) | 0 (0%) | 1 (6.7%) |
| Stage 2 | 0 (0%) | 1 (6.7%) | 3 (20%) |
| Stage 3 | 1 (16.7%) | 1 (6,7%) | 0 (0%) |
| Upper GI GVHD | | | |
| Stage 1 | 2 (33.3%) | 1 (6.7%) | 1 (6.7%) |
| Total GI_GVHD | | | |
| Any | 2 (33.3%) | 2 (13.3%) | 4 (26.7%) |

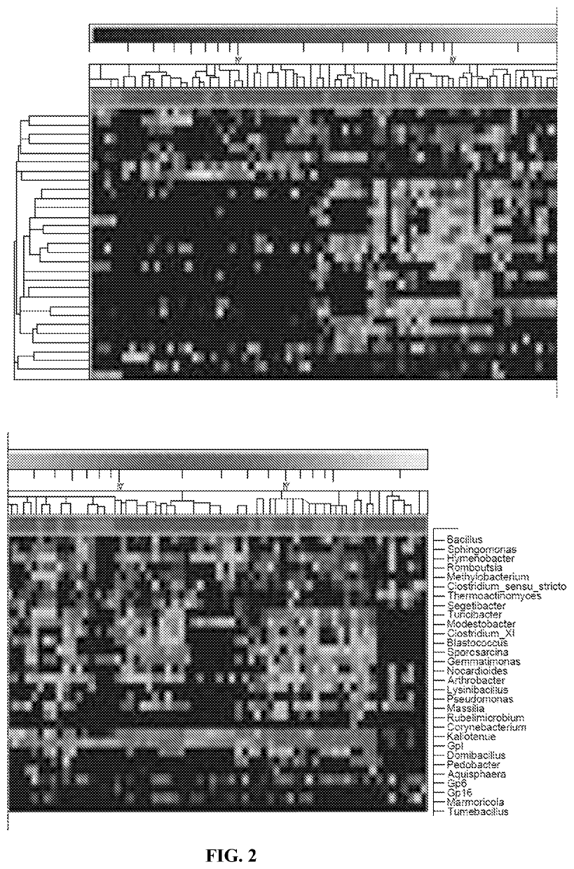

The Shanon Diversity Index was similar between the two groups at each time point tested as the microbiome analyses did not show distinct clustering between the 2 arms (FIG. 1). However, the favorable microbial profile was detected in the CBM588-treated patients. The stool microbiome profiles in the CBM588-treated patients showed reduction in the pathogenes, such as Enterobacteriaceae, Clostridium baratii and Clostridiodes difficile. (FIG. 2).

Additional cytokine profile showed reduced 1L2 levels in the Treatment arm on days 7 and 14 after HCT (p=0.006, p=0.02, respectively). Post-transplant immune reconstitution was observed by the multi-color flow cytometry analysis. Short-chain fatty acids were measured in stool samples and detailed analyses are underway.

In summary, the obtained results have demonstrated the feasibility and safety of CBM588 administration during the peri-transplant period, which was associated with an intended biological impact on the gut microbiome, and favorable early signs of GI-GVHD incidence and HCT outcomes in populations who underwent RICH CT.

The present disclosure has been described in connection with certain embodiments and examples; however, unless otherwise indicated, the claims should not be unduly limited to such specific embodiments and examples disclosed.

What is claimed is:

1. A method of treating or preventing a graft-versus-host disease (GVHD) in a subject in need thereof, said method comprising administering to said subject a therapeutically effective amount of Clostridium butyricum.

2. The method of claim 1, wherein said Clostridium butyricum is a live biotherapeutic product.

3. The method of claim 1, wherein said Clostridium butyricum live biotherapeutic product is Clostridium butyricum MIYAIRI 588 live biotherapeutic product (CBM588 LBP).

4. The method of claim 1, wherein said subject is a subject undergoing allogeneic hematopoietic stem cell transplantation.

5. The method of claim 1, wherein said subject is a cancer subject undergoing allogeneic hematopoietic stem cell transplantation.

6. The method of claim 1, wherein said cancer subject is a hematological cancer subject.

7. The method of claim 1, wherein said hematological cancer subject is leukemia subject, lymphoma subject, myeloma subject or subject with myelodysplastic syndrome.

8. The method of claim 1, wherein said subject suffers from non-cancer hematological disorder.

9. The method of claim 8, wherein said non-cancer hematological disorder is a sickle cell disorder.

10. The method of claim 1, wherein the dose of CBM588 LBP is about 50 mg per day to about 250 mg per day.

11. The method of claim 10, wherein the dose of CBM588 LBP is about 100 mg per day to about 200 mg per day.

12. The method of claim 11, wherein the dose of CBM588 LBP is about 120 mg per day to about 180 mg per day.

13. The method of claim 12 wherein the dose of CBM588 LBP is about 160 mg per day.

14. The method of claim 1, wherein CBM588 LBP is administered orally.

15. The method of claim 1, wherein CBM588 LBP is administered once a day, twice a day or three times a day.

16. The method of claim 1, wherein CBM588 LBP is administered for at least 28 days.

17. The method of claim 1, wherein CBM588 LBP is administered for at least 14 days.

18. A method of increasing gut biodiversity in a subject, said method comprising administering to said subject a therapeutically effective amount of Clostridium butyricum.

19. The method of claim 18, wherein said Clostridium butyricum is a live biotherapeutic product.

20. The method of claim 18, wherein said Clostridium butyricum live biotherapeutic product is Clostridium butyricum MIYAIRI 588 live biotherapeutic product (CBM588 LBP).

21. The method of claim 18, wherein said subject is a subject undergoing allogeneic hematopoietic stem cell transplantation.

22. The method of claim 18, wherein said subject is a cancer subject undergoing allogeneic hematopoietic stem cell transplantation.

23. The method of claim 18, wherein said cancer subject is a hematological cancer subject.

24. The method of claim 18, wherein said hematological cancer subject is leukemia subject, lymphoma subject, myeloma subject or subject with myelodysplastic syndrome.

25. The method of claim 18, wherein said subject suffers from non-cancer hematological disorder.

26. The method of claim 25, wherein said non-cancer hematological disorder is a sickle cell disorder.

27. A method of improving clinical outcome of a subject undergoing allogeneic hematopoietic stem cell transplantation, said method comprising administering to said subject a therapeutically effective amount of Clostridium butyricum.

28. The method of claim 27, wherein said Clostridium butyricum is a live biotherapeutic product.

29. The method of claim 27, wherein said Clostridium butyricum live biotherapeutic product is Clostridium butyricum MIYAIRI 588 live biotherapeutic product (CBM588 LBP).

30. The method of claim 27, wherein said subject is a subject undergoing allogeneic hematopoietic stem cell transplantation.

31. The method of claim 27, wherein said subject is a cancer subject undergoing allogeneic hematopoietic stem cell transplantation.

32. The method of claim 27, wherein said cancer subject is a hematological cancer subject.

33. The method of claim 27, wherein said hematological cancer subject is leukemia subject, lymphoma subject, myeloma subject or subject with myelodysplastic syndrome.

34. The method of claim 27, wherein said subject suffers from non-cancer hematological disorder.

35. The method of claim 34, wherein said non-cancer hematological disorder is a sickle cell disorder.

36. The method of claim 27, wherein said clinical outcome is acute GVHD or an infection.

37. A pharmaceutical composition comprising Clostridium butyricum in a dosage form.

38. The pharmaceutical composition of claim 37, wherein said Clostridium butyricum is a live biotherapeutic product.

39. The pharmaceutical composition of claim 38, wherein said Clostridium butyricum live biotherapeutic product is Clostridium butyricum MIYAIRI 588 live biotherapeutic product (CBM588 LBP).
